SEQUENCE LISTING (6 sequences)

SEQ ID NO: 1 - protein, 210 aa, synthetic construct
MAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 1273 aa, synthetic construct
  VARIANT 542: note = X optionally is N or S
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFXFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1255 aa, synthetic construct
  VARIANT 528: note = X is an N or S
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFXFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 5 - protein, 224 aa, synthetic construct
  VARIANT 224: note = X is either absent or present, and when present, is an N or S
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFX

SEQ ID NO: 6 - protein, 223 aa, synthetic construct
  VARIANT 223: note = X is either absent or present, and when present, is an N or S
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFX

(19) United States
(12) Patent Application Publication
(10) Pub. No.: US 2024/0069037 A1
(43) Pub. Date: Feb. 29, 2024

(54) METHODS FOR DETERMINING SARS-COV-2 ANTIGEN AND ANTI-SARS-COV-2 ANTIBODY IN A SAMPLE

(71) Applicant: Abbott Laboratories, Abbott Park, IL (US)

(72) Inventors: David J. Daghfal, Abbott Park, IL (US); John C. Prostko, Abbott Park, IL (US)

(21) Appl. No.: 18/344,952

(22) Filed: Jun. 30, 2023

Related U.S. Application Data
(63) Continuation of application No. PCT/US2021/065540, filed on Dec. 29, 2021.
(60) Provisional application No. 63/179,606, filed on Apr. 26, 2021.
(60) Provisional application No. 63/132,150, filed on Dec. 30, 2020.

(51) Int. Cl. G01N 33/68; G01N 33/569

(52) U.S. Cl. CPC G01N 33/6854; G01N 33/56983; G01N 2333/165

(57) ABSTRACT

Disclosed herein are methods, kits, and systems for detecting at least one type of SARS-CoV-2 antigen and at least one type of anti-SARS-CoV-2 antibody in a subject, which comprises the use of at least two different types of microparticle reagents for binding at least one type of SARS-CoV-2 antigen and at least one type of anti-SARS-CoV-2 antibody and at least two different types of detection reagents for binding each of the microparticle reagents.

### RELATED APPLICATION INFORMATION

This application is a continuation application of International Application No. PCT/US2021/065540, filed on Dec. 29, 2021, which claims priority to U.S. Application No. 63/132,150, filed on Dec. 30, 2020, and U.S. Application No. 63/179,606, filed on Apr. 26, 2021, the contents of each of which are herein incorporated by reference.

### SEQUENCE LISTING

Incorporated by reference in its entirety herein is a computer-readable nucleotide/amino acid sequence listing submitted concurrently herewith and identified as follows: One 13,723 Byte XML file named “38850-303-SQL-ST26.xml,” created on Jun. 29, 2023.

### TECHNICAL FIELD

The present disclosure relates methods, kits, systems and algorithms for detecting or determining an amount, quantity, concentration and/or level of at least one type of SARS-CoV-2 antigen and at least one type of anti-SARS-CoV-2 antibody in a biological sample from a subject.

### BACKGROUND

Viruses of the family Coronaviridae possess a single-strand, positive-sense RNA genome ranging from 26 to 32 kilobases in length (reviewed by Lu et al., The Lancet, 395:565-574 (Feb. 22, 2020)). The Coronaviridae are further subdivided (initially based on serology but now based on phylogenetic clustering) into four groups, the alpha, beta, gamma and delta coronaviruses. Coronaviruses have been identified in several avian hosts, as well as in various mammals, including camels, bats, masked palm civets, mice, dogs, and cats.

Among the several coronaviruses that are pathogenic to humans, most are associated with mild clinical symptoms, with three exceptions. Severe acute respiratory syndrome (SARS) coronavirus (SARS-CoV) is a novel betacoronavirus that emerged in Guangdong, southern China, in November 2002 and resulted in more than 8000 human infections and 774 deaths in 37 countries in 2002-03. Middle East respiratory syndrome (MERS) coronavirus (MERS-CoV) was first detected in Saudi Arabia in 2012 and was responsible for 2494 laboratory-confirmed cases of infection and 858 deaths from 2012-20. In December 2019, a cluster of pneumonia cases caused by a newly identified β-coronavirus were found to be epidemiologically-associated with the Huanan seafood market in Wuhan, China, where a number of non-aquatic animals, such as birds and rabbits were on sale before the outbreak. This coronavirus was named January 2020 by the World Health Organization (WHO) as the 2019-novel coronavirus (2019-nCov or COVID-19), and February 2020 by the International Committee as SARS-CoV-2. SARS-CoV-2 was declared a pandemic due to its rapid, uncontrolled and vast worldwide spread.

Coronavirus virions are spherical with diameters of approximately 125 nanometers, as demonstrated in studies by cryo-electron tomography and cryo-electron microscopy. A prominent feature of coronaviruses is the club-shape spike projections emanating from the surface of the virion, giving the virion the appearance of a solar corona and resulting in the name, coronaviruses. Within the envelope of the coronavirus virion is the helically-symmetrical nucleocapsid, which binds to and creates a shell around the coronavirus RNA genome. The spike (S) and nucleocapsid (N) proteins are the main immunogens of the coronavirus. The other two main structural proteins of the coronavirus particles are the membrane (M) and envelope (E) proteins. All four proteins are encoded within the 3′ end of the viral genome.

The S protein (˜150 kDa) is heavily N-linked glycosylated and utilizes an N-terminal signal sequence to gain access to the endoplasmic reticulum (ER). Homotrimers of the virus-encoding S protein make up the distinctive spike structure on the surface of the virus. In many, but not all, coronaviruses, the S protein is cleaved by a host cell furin-like protease into two separate polypeptides known as S1 and S2. S1 makes up the large receptor-binding domain of the S protein while S2 forms the stalk of the spike molecule. The trimeric S glycoprotein mediates attachment of the coronavirus virion to the host cell by interactions between the S protein and its receptor. In humans, angiotensin-converting enzyme 2 (ACE2) is the receptor for SARS-CoV. The sites of receptor binding domains (RBD) within the S1 region of a coronavirus S protein vary depending on the virus, with some having the RBD at the N-terminus of S1 (e.g., murine hepatitis virus) while others (e.g., SARS-CoV) have the RBD at the C-terminus of S1. The S-protein/receptor interaction is the primary determinant for the coronavirus to infect a host species and also governs the tissue tropism of the virus.

The M protein is the most abundant structural protein in the virion. It is a small (˜25-30 kDa) protein with 3 transmembrane domains and is believed to give the virion its shape. It has a small N-terminal glycosylated ectodomain and a much larger C-terminal endodomain that extends 6-8 nm into the viral particle.

The E protein (˜8-12 kDa) is found in small quantities within the virion. E proteins in coronaviruses are highly divergent but have a common architecture. Data suggests that the E protein is a transmembrane protein with an N-terminal ectodomain and a C-terminal endodomain that has ion channel activity. Recombinant viruses lacking the E protein are not always lethal—although this is virus-type dependent. The E protein facilitates assembly and release of the virus, but also has other functions (e.g., ion channel activity in SARS-CoV E protein is not required for viral replication but is required for pathogenesis).

The N protein is the only protein present in the nucleocapsid. It is composed of two separate domains, an N-terminal domain (NTD) and a C-terminal domain (CTD), both capable of binding RNA in vitro using different mechanisms, which may suggest that optimal RNA binding requires contributions from both domains. The N protein is heavily phosphorylated, and phosphorylation has been suggested to trigger a structural change enhancing the affinity for viral versus non-viral RNA. The N protein binds the viral genome in a beads-on-a-string type conformation. Two specific RNA substrates have been identified for N protein; the transcriptional regulatory sequences and the genomic packaging signal. The genomic packaging signal has been found to bind specifically to the second, or C-terminal RNA binding domain. The N protein also binds nsp3, a key component of the replicase complex, and the M protein. These protein interactions likely help tether the viral genome to the replicase-transcriptase complex, and subsequently package the encapsidated genome into viral particles.

In February 2020, Lu et al. reported obtaining complete and partial SARS-CoV-2 genome sequences using next-generation sequencing of bronchoalveolar lavage fluid samples and cultured isolates from nine patients from Wuhan diagnosed with viral pneumonia but negative for common respiratory pathogens. Lu et al., The Lancet, 395: 565-574 (Feb. 22, 2020). Based on their analysis, Lu et al. further reported that SARS-CoV-2 was closely related (with 88% identity) to two bat-derived severe acute respiratory syndrome (SARS)-like coronaviruses, bat-SL-CoVZC45 and bat-SL-CoVZXC21, collected in eastern China in 2018, but was more distant from SARS-CoV (about 79%) and MERS-CoV (about 50%). Additionally, Zhou et al. confirmed that SARS-CoV-2 uses the same cellular entry receptor, ACE2, as SARS-CoV. Zhou et al., Nature, 579:270-273 (March 2020).

SARS-CoV-2 primarily spreads through the respiratory tract, by droplets, respiratory secretions, and direct contact. Additionally, SARS-CoV-2 has been found in fecal swabs and blood, indicating the possibility of multiple routs of transmission. Zhang et al., Microbes 9(1):386-9 (2020). SARS-CoV-2 is highly transmissible in humans, especially in the elderly and people with underlying diseases. Symptoms can appear 2 to 14 days after exposure. Patients present with symptoms such as fever, malaise, cough, and/or shortness of breath. Most adults or children with SARS-CoV-2 infection present with mild flu-like symptoms, however, critical patients rapidly develop acute respiratory distress syndrome, respiratory failure, multiple organ failure and even death.

Because of the health risks imposed by SARS-CoV-2 transmission, there is a need for methods and kits to assess coronavirus transmission in humans, including in combination with methods to assess an anti-SARS-CoV-2 antibody and SARS-CoV-2 antigens in one or more samples obtained from a subject.

### SUMMARY

In one aspect, the present disclosure relates to a method for detecting a presence or determining an amount of at least one at least one type of anti-SARS-CoV-2 antibody or at least one type antibody fragment or variant thereof, (such as an IgA, IgG and/or IgM antibody) and at least one SARS-CoV-2 antigen or fragment or variant thereof, in a biological sample from a subject, the method comprising the steps of: a) contacting at least one biological sample, either simultaneously or sequentially, in any order, with at least one capture composition comprising at least two different types of microparticle reagents, wherein (i) the first microparticle reagent specifically binds to at least one SARS-CoV-2 antigen or fragment or variant thereof, and (ii) the second microparticle reagent specifically binds to at least one anti-SARS-CoV-2 antibody or antibody fragment or variant thereof; at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex; and b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample. In some embodiments, none of the at least two different types of microparticle reagents, at least one first detection reagent, and the at least one second detection reagent include any anti-species antibodies. In yet other embodiments, the at least two different types of microparticle reagents, the at least one first detection reagent, the at least one second detection reagent, or any combination thereof includes or contains anti-species antibodies, including, for example, antihuman IgA, IgG and/or IgM antibodies. The biological sample may be whole blood, serum, plasma, saliva, a nasal mucus specimen, an anal swab specimen, an oropharyngeal specimen, or a nasopharyngeal specimen.

In some aspects of the above method, the first microparticle reagent and the second microparticle reagent comprise at least one microparticle.

In some aspects of the above method, the at least one SARS-CoV-2 antigen detected comprises the SARS-CoV-2 spike protein or a fragment or variant thereof. In some aspects of the above method, the at least one SARS-CoV-2 antigen detected comprises the SARS-CoV-2 nucleocapsid protein or a fragment or variant thereof. In some aspects of the above method, the at least one SARS-CoV-2 antigen detected comprises the SARS-CoV-2 spike protein or fragment or variant thereof, and the SARS-CoV-2 nucleocapsid protein or fragment or variant thereof. In some aspects of the above method, the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof.

In some aspects of the above method, the second microparticle reagent comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof.

In some aspects of the above method, the first detection reagent further comprises: (i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner, thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof.

In some aspects of the above method, the second detection reagent further comprises: (i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner, thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

In some aspects of the above method, the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

In some aspects of the above method, the signal from the (1) the first complex indicates the presence or amount of anti-SARS-CoV-2 spike RBD antigen or fragment or variant in the sample; (2) the second complex indicates the presence or amount of anti-SARS-CoV-2 nucleocapsid antigen or fragment or variant in the sample; (3) the third complex indicates the presence or amount of anti-SARS-CoV-2 spike RBD antibody or antibody fragment or variant in the sample; and (4) the fourth complex indicates the presence or amount of anti-SARS-CoV-2 nucleocapsid antibody or fragment or variant in the sample.

In some aspects of the above method, the at least one type of anti-SARS-CoV-2 antibody detected is a SARS-CoV-2 IgA, SARS-CoV-2 IgG, and/or SARS-CoV-2 IgM antibody.

In other aspects of the above method, the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 infection and monitoring the subject for SARS-CoV-2 IgA, SARS-CoV-2 IgG, SARS-CoV-2 IgM antibodies and/or at least one type of SARS-CoV-2 antigen.

In some aspects of the above method, determining presence of a SARS-CoV-2 infection in a subject comprises detecting the presence of SARS-CoV-2 viral RNA using polymerase chain reaction, detecting presence of a SARS-CoV-2 viral antigen, or a combination thereof.

In some aspects of the above method, the method is performed in from about 5 to about 20 minutes, and optionally is performed in about 15 to 30 minutes.

In other aspects of the above method, the method further comprises use with at least one calibrator reagent, at least one control reagent, or at least one calibrator reagent and at least one control reagent.

In other aspects of the above method, the method is selected from the group consisting of an immunoassay or a clinical chemistry assay. In some aspects of the above method, the method is performed using single molecule detection, a lateral flow assay, or a point-of-care assay. In some aspects of the above method, it is adapted for use in an automated system or a semi-automated system.

Other aspects and aspects of the disclosure will be apparent in light of the following detailed description and accompanying figures.

### DETAILED DESCRIPTION

The present disclosure relates to methods, kits, and systems to detect the presence of or determine the amount, concentration and/or level of at least one type of SARS-CoV-2 antigen, such as at least one SARS-CoV-2 nucleocapsid protein, and at least one type of anti-SARS-CoV-2 antibody, such as an IgA, IgG and/or IgM antibody, in a sample.

The biological sample used in the methods of the present disclosure may be obtained from an asymptomatic subject or from a subject exhibiting one or more symptoms of infection with SARS-CoV-2. The methods of the present disclosure also include treating a subject identified as having a SARS-CoV-2 with one or more SARS-CoV-2 treatments and optionally, monitoring such subjects, such as before, during and/or after receiving such treatments.

In another aspect, the present disclosure relates to kits for performing such methods.

In still yet another aspect, the present disclosure relates to systems for detecting a SARS-CoV-2 in a biological sample.

Section headings as used in this section and the entire disclosure herein are merely for organizational purposes and are not intended to be limiting.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other aspects “comprising,” “consisting of” and “consisting essentially of,” the aspects or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al., J. Mol. Biol., 226: 889-896 (1992). Selective mutation at selective mutagenesis positions and at contact or hypermutation positions with an activity-enhancing amino acid residue is described in U.S. Pat. No. 6,914,128 B1.

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Anti-species antibodies” as used herein refers to an antibody, such as an IgA, IgG and/or IgM antibody, that recognize antibodies of another species of interest. For example, anti-human antibodies, e.g., anti-human IgA, IgG or IgM antibodies, recognize, respectively, other human IgA, IgG or IgM antibodies.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO—Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen to which it binds to.

As used herein, the term “coronavirus” refers to viruses that belonging to the family Coronaviridae that have a positive-sense, RNA genome ranging from 26 to 32 kilobases in length. Coronaviruses having four main structural proteins: the spike glycoprotein (S protein), the membrane protein (M protein), the envelope protein (E protein) and the nucleocapsid protein (N protein). Coronavirus can be further subdivided into four groups, alpha, beta, gamma and delta coronaviruses. Examples of alpha coronaviruses include HCoV-229E and HCoV-NL63. Examples of beta coronaviruses are HCoV-OC43, HCoV-HKU1, Middle East Respiratory Syndrome (MERS-CoV), severe acute respiratory syndrome (SARS) coronavirus (SARS-CoV) and SARS-CoV-2 (also known as 2019-nCov, COVID-19, coronavirus disease, and Coronavirus Disease 2019).

In one aspect, the present disclosure relates to β-coronaviruses. In another aspect, the β-coronaviruses are MERS-CoV, SARS-CoV and SARS-CoV-2. In still yet another aspect, the β-coronaviruses are SARS-CoV and SARC-CoV-2. In still yet another aspect, the β-coronavirus is SARS-CoV-2. The sequence of SARS-CoV-2 has been described in a variety of publications, such as, for example, Lu et al., Lancet, 395:565-574 (February 2020) and ncbi.nlm.nih.gov/genbank/sars-cov-2-seqs/, the contents of each are herein incorporated by reference.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1,” “CDR2,” and “CDR3,” for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1,” “L2,” and “L3,” or “H1,” “H2,” and “H3,” where the “L” and the “H” designate the light chain and the heavy chain regions, respectively. These regions may be referred to as “Chothia CDRs,” which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain aspects use Kabat- or Chothia-defined CDRs.

“Component,” “components,” or “at least one component,” refer generally to a microparticle reagent (such as a first microparticle reagent) or a specific binder and related microparticle, a detection reagent or a specific binding partners and a detectable label, a calibrator, a control, a sensitivity panel, a container, a buffer, a diluent (including an assay specific diluent), a salt, an enzyme, a co-factor for an enzyme, a pretreatment reagent/solution, a substrate (e.g., as a solution), a stop solution, and the like that can be included in a kit for assay of a test sample, such as a patient nasal mucus specimen, an anal swab specimen, saliva, oropharyngeal specimens, nasopharyngeal specimens, urine, saliva, whole blood, serum or plasma sample (e.g., as per “Sample” below) in accordance with the methods described herein and other methods known in the art. Some components can be in solution or lyophilized for reconstitution for use in an assay

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like). As used herein, a “control subject” relates to a subject or subjects that has not been infected with a coronavirus, such as, a β-coronavirus (such as SARS-CoV or SARS-CoV-2) or been exposed to any subject that has had a coronavirus, such as a β-coronavirus (such as SARS-CoV or SARS-CoV-2).

As used herein, the term “control line” or “control zone” is a region of a test strip in which a label can be observed to shift location, appear, change color, or disappear to indicate that an assay performed correctly. Detection or observation of the control zone (e.g., of a control line) may be done by any convenient means, depending upon the particular choice of label, especially, for example but not limited to, visually, fluorescently, by reflectance, radiographically, and the like. As will be described, the label may or may not be applied directly to the control zone, depending upon the design of the control being used.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Determined by an assay” is used herein to refer to the determination of a reference level by any appropriate assay. The determination of a reference level may, in some aspects, be achieved by an assay of the same type as the assay that is to be applied to the sample from the subject (for example, by an immunoassay, clinical chemistry assay, a single molecule detection assay, protein immunoprecipitation, immunoelectrophoresis, a point-of-care assay, chemical analysis, SDS-PAGE and Western blot analysis, or protein immunostaining, electrophoresis analysis, a protein assay, or a competitive binding assay. The determination of a reference level may, in some aspects, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure. For example, a set of training samples comprising samples obtained from subjects known to have been infected by a coronavirus, such as a β-coronavirus, and samples obtained from human subjects known not to have been infected with a coronavirus, such as a β-coronavirus, or been exposed to a subject that has been infected with a coronavirus, such as a β-coronavirus, may be used to obtain assay-specific reference levels. It will be understood that a reference level “determined by an assay” and having a recited level of “sensitivity” and/or “specificity” is used herein to refer to a reference level which has been determined to provide a method of the recited sensitivity and/or specificity when said reference level is adopted in the methods of the disclosure. It is well within the ordinary skill of one in the art to determine the sensitivity and specificity associated with a given reference level in the methods of the disclosure, for example by repeated statistical analysis of assay data using a plurality of different possible reference levels.

Practically, when discriminating between a subject as having been infected by a coronavirus, such as a β-coronavirus (such as SARS-CoV or SARS-CoV-2) or not having been infected by a coronavirus, such as a β-coronavirus (such as SARS-CoV or SARS-CoV-2), the skilled person will balance the effect of raising a cutoff on sensitivity and specificity. Raising or lowering a cutoff will have a well-defined and predictable impact on sensitivity and specificity, and other standard statistical measures. It is well known that raising a cutoff will improve specificity but is likely to worsen sensitivity (proportion of those with disease who test positive). In contrast, lowering a cutoff will improve sensitivity but will worsen specificity (proportion of those without disease who test negative). The ramifications for detecting or measuring a coronavirus, such as a β-coronavirus (such as SARS-CoV or SARS-CoV-2), will be readily apparent to those skilled in the art. In discriminating whether a subject has or has not been infected by a coronavirus, such as a β-coronavirus (such as SARS-CoV or SARS-CoV-2), the higher the cutoff, specificity improves as more true negatives (i.e., subjects not having been infected by a coronavirus, such as β-coronavirus (such as SARS-CoV or SARS-CoV-2)) are distinguished from those having been infected by a coronavirus, such as a β-coronavirus (such as SARS-CoV or SARS-CoV-2). But at the same time, raising the cutoff decreases the number of cases identified as positive overall, as well as the number of true positives, so the sensitivity must decrease. Conversely, the lower the cutoff, sensitivity improves as more true positives (i.e., subjects having been infected with a coronavirus, such as a β-coronavirus (such as SARS-CoV or SARS-CoV-2)) are distinguished from those who have not been infected (e.g., do not have) with a coronavirus, such as a β-coronavirus (such as SARS-CoV or SARS-CoV-2). But at the same time, lowering the cutoff increases the number of cases identified as positive overall, as well as the number of false positives, so the specificity must decrease.

Generally, a high sensitivity value helps one of skill rule out disease or condition (such as infection with a coronavirus, such as a β-coronavirus (such as SARS-CoV or SARS-CoV-2)), and a high specificity value helps one of skill rule in disease or condition. Whether one of skill desires to rule out or rule in disease depends on what the consequences are for the patient for each type of error. Accordingly, one cannot know or predict the precise balancing employed to derive a test cutoff without full disclosure of the underlying information on how the value was selected. The balancing of sensitivity against specificity and other factors will differ on a case-by-case basis. This is why it is sometimes preferable to provide alternate cutoff (e.g., reference) values so a physician or practitioner can choose.

“Dual-specific antibody” is used herein to refer to a full-length antibody that can bind two different antigens (or epitopes) in each of its two binding arms (a pair of HC/LC) (see PCT International Application WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule, but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT International Application WO 2007/024715, U.S. Pat. No. 7,612,181, and Wu et al., Nature Biotech., 25: 1290-1297 (2007). An example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In another embodiment, a DVD-Ig binding protein not only binds the same target molecules bound by its parental monoclonal antibodies, but also possesses one or more desirable properties of one or more of its parental monoclonal antibodies. Such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of nucleocapsid protein, spike protein or nucleocapsid protein and spike protein from a coronavirus, such as a β-coronavirus (such as SARS-CoV or SARS-CoV-2). Non-limiting examples of a DVD-Ig binding protein include a DVD-Ig binding protein that binds one or more epitopes of a nucleocapsid protein, spike protein, or nucleocapsid protein and spike protein of a β-coronavirus (such as SARS-CoV or SARS-CoV-2), a DVD-Ig binding protein that binds an epitope of a human nucleocapsid protein, spike protein, or nucleocapsid protein and spike protein of a β-coronavirus (such as SARS-CoV or SARS-CoV-2) and an epitope of a nucleocapsid protein, spike protein, or nucleocapsid protein and spike protein of a β-coronavirus (such as SARS-CoV or SARS-CoV-2) of another species (for example, mouse, rat, bat, etc.), and a DVD-Ig binding protein that binds an epitope of a human β-coronavirus (such as SARS-CoV or SARS-CoV-2) and an epitope of another target molecule.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international IMMUNOGENETICS® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

The term “fusion protein” as used herein relates to a protein or polypeptide comprising at least one first protein or polypeptide joined or linked to at least one second protein or polypeptide. In some aspects, the at least one protein or polypeptide is joined or linked to at least one second protein or polypeptide through one or more linking peptide sequences. An example of a fusion protein is a chimeric protein. A fusion protein can be created using routine techniques known in the art such as recombinant DNA technology, through joining or linking of two or more genes that originally coded for separate proteins. Thus, a fusion protein may comprise a multimer of different or identical binding proteins which are expressed as a single, linear polypeptide.

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some aspects, a humanized antibody contains the light chain as well as at least the variable domain of a heavy chain. The antibody also may include the CH1, hinge, CH2, CH3, and CH4 regions of the heavy chain. In some aspects, a humanized antibody only contains a humanized light chain. In some aspects, a humanized antibody only contains a humanized heavy chain. In specific aspects, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence. As used herein, “isolated polypeptide” refers to a polypeptide (e.g., of recombinant, synthetic or chemical original or a combination thereof), that, by virtue of its origin, the isolated polypeptide is not associated with all or a portion of a polypeptide and/or other protein(s) with which the “isolated polypeptide” is found in nature; is operably linked to a polypeptide and/or protein that it is not linked to in nature; or does not occur in nature as part of a larger sequence. When associated with a particular species, virus or strain (e.g., “O-coronavirus isolated polypeptide”), the isolated polypeptide optionally can be made by recombinant means rather than by isolation from in vivo.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 1251, 1311, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, MI). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. Pat. No. 7,906,293. Acridinium-9-carboxylate aryl esters can be dissolved in any suitable solvent, such as degassed anhydrous N,N-dimethylformamide (DMF) or aqueous sodium cholate.

“Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Multivalent binding protein” is used herein to refer to a binding protein comprising two or more antigen binding sites (also referred to herein as “antigen binding domains”). A multivalent binding protein is preferably engineered to have three or more antigen binding sites, and is generally not a naturally occurring antibody. The term “multispecific binding protein” refers to a binding protein that can bind two or more related or unrelated targets, including a binding protein capable of binding two or more different epitopes of the same target molecule.

“Nucleocapsid protein” or “N” protein as used interchangeably herein, refers to one of four main structural proteins of a coronavirus. The N protein is the only protein present in the nucleocapsid. It is composed of two separate domains, an N-terminal domain (NTD) and a C-terminal domain (CTD), both capable of binding RNA in vitro using different mechanisms, which may suggest that optimal RNA binding requires contributions from both domains. For example, in SARS-CoV-2, the NTD can be found at amino acids 1 to 209 of SEQ ID NO: 1. In SARS-CoV, the NTD can be found at amino acids 1 to 210 of SEQ ID NO: 2. For example, in SARS-CoV-2, the CTD can be found at amino acids 210 to 419 of SEQ ID NO: 1. In SARS-CoV, the CTD can be found at amino acids 211 to 422 of SEQ ID NO: 2.

In some aspects described herein, a nucleocapsid protein is at least a portion (e.g., at least 5 amino acids or more) or the entirety of a nucleocapsid protein from a SARS-CoV-2 strain of β-coronavirus comprising the sequence of SEQ ID NO:1, and is referred to here as a “CTD peptide,” “Nc-CTD peptide,” or “Nc-Cbt peptide”.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). Examples of point-of-care devices include those produced by Abbott Laboratories (Abbott Park, IL) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA).

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low,” “medium,” or “high” levels), can be used. Multiple calibrators (i.e., more than one calibrator or a varying amount of calibrator(s)) can be used in conjunction to comprise a “sensitivity panel.”

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-IG®s, and other antibodies as described herein (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Reference level” as used herein refers to an assay cutoff value (or level) that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). As used herein, the term “cutoff” refers to a limit (e.g., such as a number) above which there is a certain or specific clinical outcome and below which there is a different certain or specific clinical outcome.

This disclosure provides exemplary reference levels. However, it is well-known that reference levels may vary depending on the nature of the immunoassay (e.g., capture and detection reagents employed, reaction conditions, sample purity, etc.) and that assays can be compared and standardized. It further is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays.

“Sample,” “test sample,” “specimen,” “sample from a subject,” “biological sample,” and “patient sample” may be used interchangeably herein to refer to a sample of blood, such as whole blood (including for example, capillary blood, venous blood, dried blood spot, etc.), saliva, tissue, urine, serum, plasma, amniotic fluid, lower respiratory specimens such as, but not limited to, sputum, endotracheal aspirate or bronchoalveolar lavage, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Additionally, the sample can be a nasopharyngeal or oropharyngeal sample obtained using one or more swabs that, once obtained, is placed in a sterile tube containing a virus transport media (VTM) or universal transport media (UTM), for testing. Further, the sample can be a nasal mucus specimen.

A variety of cell types, tissue, or bodily fluid may be utilized to obtain a sample. Such cell types, tissues, and fluid may include sections of tissues such as biopsy and autopsy samples, nasal mucus specimens, anal swab specimens, oropharyngeal specimens, nasopharyngeal specimens, frozen sections taken for histologic purposes, blood (such as whole blood, dried blood spots, etc.), plasma, serum, red blood cells, platelets, interstitial fluid, cerebrospinal fluid, etc. Cell types and tissues may also include lymph fluid, cerebrospinal fluid, or any fluid collected by aspiration. A tissue or cell type may be provided by removing a sample of cells from a human and a non-human animal, but also can be accomplished by using previously isolated cells (e.g., isolated by another person, at another time, and/or for another purpose). Archival tissues, such as those having treatment or outcome history, may also be used. Protein or nucleotide isolation and/or purification may not be necessary. In some aspects, the sample is a whole blood sample. In some aspects, the sample is a capillary blood sample. In some aspects, the sample is a dried blood spot. In some aspects, the sample is a serum sample. In yet other aspects, the sample is a plasma sample. In some aspects, the sample is an oropharyngeal specimen. In other aspects, the sample is a saliva sample. In yet other aspects, the sample is an anal swab specimen. In other aspects, the sample is a nasopharyngeal specimen. In other aspects, the sample is sputum. In other aspects, the sample is endotracheal aspirate. In still yet other aspects, the sample is bronchoalveolar lavage. In yet other aspects, the sample is a nasal mucus specimen.

“Sensitivity” of an assay as used herein refers to the proportion of subjects for whom the outcome is positive that are correctly identified as positive (e.g., correctly identifying those subjects with a disease or medical condition for which they are being tested). For example, this may include correctly identifying subjects as having been infected with a coronavirus, such as a β-coronavirus (such as SARS-CoV or SARS-CoV-2), from those who have not been infected with a coronavirus, such as a β-coronavirus (such as SARS-CoV or SARS-CoV-2).

“Specificity” of an assay as used herein refers to the proportion of subjects for whom the outcome is negative that are correctly identified as negative (e.g., correctly identifying those subjects who do not have a disease or medical condition for which they are being tested). For example, this may include correctly identifying subjects having been infected with a coronavirus, such as a β-coronavirus (such as SARS-CoV or SARS-CoV-2), from those who have not been infected with a coronavirus, such as a β-coronavirus (such as SARS-CoV or SARS-CoV-2).

“Series of calibrating compositions” refers to a plurality of compositions comprising a known concentration of the analytes, such as one or more antibodies (such as anti-SARS-CoV-2 (IgG, IgA, or IgM) antibodies)), or polypeptides (such as one or more peptides derived from SARS-CoV-2) wherein each of the compositions differs from the other compositions in the series by the concentration of the analytes.

As used herein the term “single molecule detection” refers to the detection and/or measurement of a single molecule of an analyte in a test sample at very low levels of concentration (such as pg/mL or femtogram/mL levels). A number of different single molecule analyzers or devices are known in the art and include nanopore and nanowell devices. Examples of nanopore devices are described in PCT International Application WO 2016/161402, which is hereby incorporated by reference in its entirety. Examples of nanowell device are described in PCT International Application WO 2016/161400, which is hereby incorporated by reference in its entirety.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner, or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

“Specific binding partner” or “Specific binding member,” as used interchangeable herein, is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Spike protein” or “S” protein as used interchangeably herein refers to one of four main structural proteins of a coronavirus. The spike protein is heavily N-linked glycosylated and utilizes an N-terminal signal sequence to gain access to the endoplasmic reticulum (ER). Homotrimers of the virus-encoding S protein make up the distinctive spike structure on the surface of the virus. In many coronaviruses, the S protein is cleaved by a host cell furin-like protease into two separate polypeptides noted S1 and S2. S1 makes up the large receptor-binding domain (RBD) of the S protein while S2 forms the stalk of the spike molecule. The trimeric S glycoprotein mediates attachment of the coronavirus virion to the host cell by interactions between the S protein and its receptor. In humans, angiotensin-converting enzyme 2 (ACE2) is the receptor for SARS-CoV and SARS-CoV-2.

“Subject” and “patient” as used herein interchangeably refers to any vertebrate, including, but not limited to, a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, chimpanzee, etc.) and a human). In some aspects, the subject may be a human, a non-human primate or a cat. In some aspects, the subject is a human. The subject or patient may be undergoing other forms of treatment. In some aspects, the subject is a human that may be undergoing other forms of treatment. In some aspects, the subject is suspected to have, have had or has been exposed to a subject that has had or tested positive for infection with a coronavirus, such as a β-coronavirus (such as SARS-CoV or SARS-CoV-2). In other aspects, the subject is completely asymptomatic and does not exhibit any symptoms of a coronavirus, such as a β-coronavirus (such as SARS-CoV or SARS-CoV-2), and may or may not have been exposed to a subject that has or has been exposed or infected with a coronavirus, such as a β-coronavirus (such as SARS-CoV or SARS-CoV-2).

As used herein, a “system” refers to a plurality of real and/or abstract elements operating together for a common purpose. In some aspects, a “system” is an integrated assemblage of hardware and/or software elements. In some aspects, each component of the system interacts with one or more other elements and/or is related to one or more other elements. In some aspects, a system refers to a combination of components and software for controlling and directing methods.

As used herein, the term “test strip” can include one or more bibulous or non-bibulous materials. If a test strip comprises more than one material, the one or more materials are preferably in fluid communication. One material of a test strip may be overlaid on another material of the test strip, such as for example, filter paper overlaid on nitrocellulose. Alternatively or in addition, a test strip may include a region comprising one or more materials followed by a region comprising one or more different materials. In this case, the regions are in fluid communication and may or may not partially overlap one another. Suitable materials for test strips include, but are not limited to, materials derived from cellulose, such as filter paper, chromatographic paper, nitrocellulose, and cellulose acetate, as well as materials made of glass fibers, nylon, dacron, PVC, polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH, salt concentration, or specific gravity of an applied sample to optimize test conditions.

The material or materials can be a single structure such as a sheet cut into strips or it can be several strips or particulate material bound to a support or solid surface such as found, for example, in thin-layer chromatography and may have an absorbent pad either as an integral part or in liquid contact. The material also can be a sheet having lanes thereon, capable of spotting to induce lane formation, wherein a separate assay can be conducted in each lane. The material can have a rectangular, circular, oval, triangular, or other shape provided that there is at least one direction of traversal of a test solution by capillary migration. Other directions of traversal may occur such as in an oval or circular piece contacted in the center with the test solution. However, the main consideration is that there be at least one direction of flow to a predetermined site.

The support for the test strip, where a support is desired or necessary, will normally be water insoluble, frequently non-porous and rigid but may be elastic, usually hydrophobic, and porous and usually will be of the same length and width as the strip but may be larger or smaller. The support material can be transparent, and, when a test device of the present technology is assembled, a transparent support material can be on the side of the test strip that can be viewed by the user, such that the transparent support material forms a protective layer over the test strip where it may be exposed to the external environment, such as by an aperture in the front of a test device. A wide variety of non-mobilizable and non-mobilizable materials, both natural and synthetic, and combinations thereof, may be employed provided only that the support does not interfere with the capillary action of the material or materials, or non-specifically bind assay components, or interfere with the signal producing system. Illustrative polymers include polyethylene, polypropylene, poly(4-methylbutene), polystyrene, polymethacrylate, poly(ethylene terephthalate), nylon, poly(vinyl butyrate), glass, ceramics, metals, and the like. Elastic supports may be made of polyurethane, neoprene, latex, silicone rubber and the like.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Variant” is used herein to describe a peptide or polypeptide that differs from a reference peptide or polypeptide in amino acid sequence by the insertion, deletion, or conservative substitution of amino acids, but retains at least one biological activity. Representative examples of “biological activity” include the ability to be bound by a specific antigen or antibody, or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically-reactive fragment of an anti-analyte antibody that differs from the corresponding fragment of anti-analyte antibody in amino acid sequence but is still antigenically reactive and can compete with the corresponding fragment of anti-analyte antibody for binding with the analyte. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid,” which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Methods for Detecting the Presence of or Determining the Amount of at Least One Type of Anti-SARS-CoV-2 Antibody and at Least One Type of SARS-CoV-2 Antigen in a Biological Sample from a Subject

The present disclosure relates to methods for (a) detecting the presence of at least one type of anti-SARS-CoV-2 antibody or fragment thereof and at least one type of SARS-CoV-2 antigen or fragment or variant thereof (e.g., SARS-CoV-2 nucleocapsid, SARS-CoV-2 spike protein, and any combinations thereof (including any combination of fragments thereof)); and/or (b) determining or measuring the amount or level of at least one type of anti-SARS-CoV-2 antibody or fragment thereof and at least one type of SARS-CoV-2 antigen or fragment or variant thereof, in a biological sample (e.g., a single biological sample or multiple biological samples) obtained from a subject (e.g., such as a human, a non-human primate, a cat, etc.). In still yet other embodiments, the methods described herein can be used as an aid in the diagnosis of a SARS-CoV-2 infection.

It is understood that variants in the nucleocapsid and spike proteins (namely, SARS-CoV-2 variant polypeptides) are being compiled and reported continually by researchers worldwide as the SARS-CoV-2 virus continues to mutate over time. Any of the mutations (e.g., one or more insertions, substitutions and/or deletions) in these variants (e.g., SARS-CoV-2 variant polypeptides) can be used for the nucleocapsid protein or spike or RBD protein, or fragment thereof, as described herein, either alone, or in combination with the changes in the variants described in further detail below, so long as these mutations do not negate an ability to detect the presence of or determine the amount of at least one type of anti-SARS-CoV-2 antibody or fragment and at least one type of SARS-CoV-2 antigen or fragment or variant thereof in a biological sample obtained from a subject.

In some aspects, for example, the methods described herein can be used in conjunction with clinical presentation and other laboratory tests to aid in the diagnosis of SARS-CoV-2 infection in a subject (e.g., who may or may not exhibit signs and/or symptoms of infection, or be suspected of having SARS-CoV-2). It should be understood that a “negative” result obtained using the methods described herein (e.g., where the presence of at least one type of anti-SARS-CoV-2 antibody or fragment thereof and at least one type of SARS-CoV-2 antigen or fragment or variant thereof is not detected and/or the amount, level or concentration of at least one type of anti-SARS-CoV-2 antibody and at least one type of SARS-CoV-2 antigen cannot be determined or is below a predetermined level or cutoff) does not rule out prior or current infection with SARS-CoV-2, particularly in those subjects who have been in contact with the virus (e.g., health care workers). Typically, such subjects might receive follow-up or further testing with a molecular diagnostic to further rule out infection in said individuals.

In some embodiments, the detection in samples of at least one type of anti-SARS-CoV-2 antibody or fragment thereof and at least one type SARS-CoV-2 antigen or fragment or variant thereof is an indication of current or past presence in the subject of the virus. In some embodiments, the methods comprise: contacting at least one biological sample, either simultaneously or sequentially, in any order, with at least one capture composition comprising at least two different types of microparticle reagents, wherein (i) the first microparticle reagent specifically binds to at least one type of SARS-CoV-2 antigen or fragment or variant thereof (e.g., SARS-CoV-2 nucleocapsid, SARS-CoV-2 spike protein, and any combinations thereof (including any combination of fragments thereof)), and (ii) the second microparticle reagent specifically binds to at least one type of anti-SARS-CoV-2 antibody or antibody fragment or variant thereof (e.g., an anti-SARS-CoV-2 IgA antibody, an anti-SARS-CoV-2 IgM antibody, an anti-SARS-CoV-2 IgG antibody and any combination thereof (including any combination of antibody fragments thereof)), and at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex. In some embodiments, none of the at least two different types of microparticle reagents, at least one first detection reagent, and the at least one second detection reagent include or contain any anti-species antibodies. In still yet other embodiments, the at least two different types of microparticle reagents, the at least one first detection reagent, the at least one second detection reagent, or any combination thereof includes or contains anti-species antibodies, including, for example, anti-human IgA, anti-human IgG, anti-human IgM antibodies or any combination thereof. In select embodiments, the at least two different types of microparticle reagents, the at least one first detection agent, at least one second detection reagent or any combination thereof includes or contains anti-species antibodies, including, for example, anti-human IgA, IgG, IgM antibodies or any combination thereof. In one aspect, the antibody used can be an anti-species IgA (e.g., anti-human-IgA IgG), an anti-species IgG (e.g., anti-human-IgG IgG) antibody, an anti-species IgM (e.g., anti-human-IgM IgG) antibody, anti-species IgA (e.g., anti-human-IgA IgG) and anti-species IgG (e.g., anti-human-IgG IgG), anti-species IgA (e.g., anti-human-IgA IgG) and anti-species IgM (e.g., anti-human-IgM IgG), an anti-species IgG (e.g., anti-human-IgG IgG) and anti-species IgM (e.g., anti-human-IgM IgG), or anti-species IgA (e.g., anti-human-IgA IgG), anti-species IgG (e.g., anti-human-IgG IgG), anti-species IgM (e.g., anti-human IgM IgG) antibody. For example, in some aspects, the antibody used can be a mouse IgA antibody, a mouse IgG antibody, a mouse IgM antibody, a mouse IgA and mouse IgG antibody, a mouse IgA antibody and a mouse IgM antibody, a mouse IgG and mouse IgM antibody, or a mouse IgA, mouse IgG and a mouse IgM antibody, a rat IgA antibody, a rat IgG antibody, a rat IgM antibody, a rat IgA and rat IgG antibody, a rat IgA antibody and a rat IgM antibody, a rat IgG and rat IgM antibody, or a rat IgA, rat IgG and a rat IgM antibody, a rabbit IgA antibody, a rabbit IgG antibody, a rabbit IgM antibody, a rabbit IgA and rabbit IgG antibody, a rabbit IgA antibody and a rabbit IgM antibody, a rabbit IgG and rabbit IgM antibody, or a rabbit IgA, rabbit IgG and a rabbit IgM antibody, a goat IgA antibody, a goat IgG antibody, a goat IgM antibody, a goat IgA and goat IgG antibody, a goat IgA antibody and a goat IgM antibody, a goat IgG and goat IgM antibody, or a goat IgA, goat IgG and a goat IgM antibody, a sheep IgA antibody, a sheep IgG antibody, a sheep IgM antibody, a sheep IgA and sheep IgG antibody, a sheep IgA antibody and a sheep IgM antibody, a sheep IgG and sheep IgM antibody, or a sheep IgA, sheep IgG and a sheep IgM antibody, a non-human primate IgA antibody, a non-human primate IgG antibody, a non-human primate IgM antibody, a non-human primate IgA and non-human primate IgG antibody, a non-human primate IgA antibody and a non-human primate IgM antibody, a non-human primate IgG and non-human primate IgM antibody, or a non-human primate IgA, non-human primate IgG and a non-human primate IgM antibody, a human IgA antibody, a human IgG antibody, a human IgM antibody, a human IgA and human IgG antibody, a human IgA antibody and a human IgM antibody, a human IgG and human IgM antibody, or a human IgA, human IgG and a human IgM antibody. In one aspect, the at least two different types of microparticle reagents, the at least one first detection reagent, the at least one second detection reagent, or any combination thereof can be anti-human IgG (mouse monoclonal) antibody available in the ARCHITECT®/Alinity® I Rubella IgG assay (Abbott Laboratories, Abbott Park, IL), although any other commercially available anti-species IgG (e.g., anti-human-IgG IgG) antibody can be used. In another aspect, the at least two different types of microparticle reagents, the at least one first detection reagent, the at least one second detection reagent, or any combination thereof can be the murine anti-human IgM antibody available in the ARCHITECT®/Alinity® I Rubella IgM assay (Abbott Laboratories, Abbott Park, IL), although any other commercially available anti-species IgG (e.g., anti-human-IgG IgG) antibody can be used.

In some embodiments, the first microparticle reagent specifically binds to the SARS-CoV-2 nucleocapsid protein or a fragment or variant thereof. In some embodiments, first microparticle reagent specifically binds to the SARS-CoV-2 spike protein or a fragment or variant thereof. In some embodiments, the first microparticle reagent specifically binds to the receptor binding domain (RBD) of the SARS-CoV-2 spike protein or a fragment of the SARS-CoV-2 spike protein comprising the RBD. In some embodiments, the first microparticle reagent specifically binds to the SARS-CoV-2 nucleocapsid protein and the SARS-CoV-2 spike protein or any fragment or variant thereof.

In some embodiments, the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof.

In some embodiments, the first microparticle reagent comprises a single type of microparticle comprising one or more specific binding partners (e.g., for binding to the SARS-CoV-2 nucleocapsid protein, the SARS-CoV-2 spike protein, the SARS-CoV-2 nucleocapsid protein and the SARS-CoV-2 spike protein or any fragments or variants thereof). In some embodiments, the first microparticle reagent comprises more than one type of microparticle. For example, when first microparticle reagent specifically binds to the SARS-CoV-2 nucleocapsid protein and the SARS-CoV-2 spike protein, one type of microparticle may be configured to bind the SARS-CoV-2 nucleocapsid protein and a second type of microparticle may be configured to bind to the SARS-CoV-2 spike protein.

In some embodiments, the second microparticle reagent specifically binds to at least one type of anti-SARS-CoV-2 antibody or antibody fragment or variant thereof (e.g., anti-SARS-CoV-2 IgA antibody, an anti-SARS-CoV-2 IgM antibody, an anti-SARS-CoV-2 IgG antibody or any combination thereof (including any combination of antibody fragments thereof)). In some embodiments, the second microparticle reagent specifically binds to an anti-SARS-CoV-2 nucleocapsid protein antibody or antibody fragment or variant thereof. In some embodiments, the second microparticle reagent specifically binds to an anti-SARS-CoV-2 spike protein antibody or antibody fragment or variant thereof. In some embodiments, the second microparticle reagent specifically binds to an anti-SARS-CoV-2 spike protein antibody or antibody fragment or variant thereof. In some embodiments, the second microparticle reagent specifically binds to an anti-SARS-CoV-2 nucleocapsid protein antibody or antibody fragment or variant thereof and an anti-SARS-CoV-2 spike protein antibody or antibody fragment or variant thereof.

In some embodiments, the second microparticle reagent-comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof.

In some embodiments, the second microparticle reagent comprises a single type of microparticle comprising one or more specific binding partners (e.g., for binding an anti-SARS-CoV-2 nucleocapsid protein antibody, an anti-SARS-CoV-2 spike protein antibody, or an anti-SARS-CoV-2 nucleocapsid protein antibody and an anti-SARS-CoV-2 spike protein antibody (including any combination of any antibody fragments or variants thereof)). In some embodiments, the second microparticle reagent comprises more than one type of microparticle. For example, when the second microparticle reagent specifically binds to an anti-SARS-CoV-2 nucleocapsid protein antibody or antibody fragment or variant thereof and an anti-SARS-CoV-2 spike protein antibody or antibody fragment or variant thereof, one type of microparticle may be configured to bind the anti-SARS-CoV-2 nucleocapsid protein antibody or antibody fragment or variant thereof and a second type of microparticle may be configured to bind to the anti-SARS-CoV-2 spike protein antibody or antibody fragment or variant thereof.

In some embodiments, the at least one first detection reagent binds to at least one type of SARS-CoV-2 antigen or fragment or variant thereof at a different location than that of the specific binding partner in the first microparticle reagent. In some embodiments, the at least one first detection reagent binds to the SARS-CoV-2 spike protein or fragment or variant thereof at a different location than that of the specific binding partner in the first microparticle reagent. In some embodiments, the at least one first detection reagent binds to the SARS-CoV-2 spike protein RBD or fragment or variant thereof at a different location than that of the specific binding partner in the first microparticle reagent. In some embodiments, the at least one first detection reagent binds to the SARS-CoV-2 nucleocapsid protein or fragment or variant thereof at a different location than that of the specific binding partner in the first microparticle reagent.

In some embodiments, the first detection reagent further comprises: (i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner.

In some embodiments, the at least one second detection reagent binds to at least one type of anti-SARS-CoV-2 antibody or antibody fragment or variant thereof at a different location than that of the specific binding partner in the second microparticle reagent. In some embodiments, the at least one first detection reagent binds to the anti-SARS-CoV-2 spike protein antibody or antibody fragment or variant thereof at a different location than that of the specific binding partner in the second microparticle reagent. In some embodiments, the at least one first detection reagent binds to the anti-SARS-CoV-2 spike protein RBD antibody or antibody fragment or variant thereof at a different location than that of the specific binding partner in the second microparticle reagent. In some embodiments, the at least one first detection reagent binds to the anti-SARS-CoV-2 nucleocapsid protein antibody or antibody fragment or variant thereof at a different location than that of the specific binding partner in the second microparticle reagent.

In some embodiments, the second detection reagent further comprises: (i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment thereof at a different location then the at least one fourth specific binding partner

First and Fifth Specific Binding Partners. In some embodiments, the first specific binding partner and the fifth specific binding partner are each specific for an epitope from the SARS-CoV-2 spike protein or fragment or variant thereof. The spike protein comprises an N-terminal (S1) subunit, which contains the roughly 200-residue receptor binding domain (RBD), and a C-terminal subunit (S2), which contains the fusion peptide, heptad repeats 1 and 2, the transmembrane anchor, and the intracellular tail. In some embodiments, the first specific binding partner and fifth specific binding partner specific binding partner are each specific for an epitope in the C-terminal domain. In some embodiments, the first specific binding partner and fifth specific binding partner specific binding partner are each specific for an epitope in the N-terminal domain. In some embodiments, the first specific binding partner is specific for an epitope in the N-terminal domain and the fifth specific binding partner specific binding partner are each specific for an epitope in the C-terminal domain. In some embodiments, the first specific binding partner is specific for an epitope in the C-terminal domain and the fifth specific binding partner specific binding partner are each specific for an epitope in the N-terminal domain. In some embodiments, the first specific binding partner and fifth specific binding partner are each specific for an epitope in the receptor binding domain of the spike protein.

In another aspect, the epitope from the SARS-CoV-2 spike protein or fragment or variant thereof against which the first specific binding partner and/or the fifth specific binding partner is directed can have a length of about 5 amino acids to about 500 amino acids, about 10 amino acids to about 500 amino acids, about 15 amino acids to about 500 amino acids, about 20 amino acids to about 500 amino acids, about 26 amino acids to about 500 amino acids, about 30 amino acids to about 500 amino acids, about 40 amino acids to about 500 amino acids, about 50 amino acids to about 500 amino acids, about 60 amino acids to about 500 amino acids, about 70 amino acids to about 500 amino acids, about 75 amino acids to about 500 amino acids, about 80 amino acids to about 500 amino acids, about 90 amino acids to about 500 amino acids, about 100 amino acids to about 500 amino acids, about 5 amino acids to about 400 amino acids, about 10 amino acids to about 400 amino acids, about 15 amino acids to about 400 amino acids, about 20 amino acids to about 400 amino acids, about 26 amino acids to about 400 amino acids, about 30 amino acids to about 400 amino acids, about 40 amino acids to about 400 amino acids, about 50 amino acids to about 400 amino acids, about 60 amino acids to about 400 amino acids, about 70 amino acids to about 400 amino acids, about 75 amino acids to about 400 amino acids, about 80 amino acids to about 400 amino acids, about 90 amino acids to about 400 amino acids, about 100 amino acids to about 400 amino acids, about 5 amino acids to about 300 amino acids, about 10 amino acids to about 300 amino acids, about 15 amino acids to about 300 amino acids, about 20 amino acids to about 300 amino acids, about 26 amino acids to about 300 amino acids, about 30 amino acids to about 300 amino acids, about 40 amino acids to about 300 amino acids, about 50 amino acids to about 300 amino acids, about 60 amino acids to about 300 amino acids, about 70 amino acids to about 300 amino acids, about 75 amino acids to about 300 amino acids, about 80 amino acids to about 300 amino acids, about 90 amino acids to about 300 amino acids, about 100 amino acids to about 300 amino acids, about 5 amino acids to about 200 amino acids, about 10 amino acids to about 200 amino acids, about 15 amino acids to about 200 amino acids, about 20 amino acids to about 200 amino acids, about 26 amino acids to about 200 amino acids, about 30 amino acids to about 200 amino acids, about 40 amino acids to about 200 amino acids, about 50 amino acids to about 200 amino acids, about 60 amino acids to about 200 amino acids, about 70 amino acids to about 200 amino acids, about 75 amino acids to about 200 amino acids, about 80 amino acids to about 200 amino acids, about 90 amino acids to about 200 amino acids, about 100 amino acids to about 200 amino acids, about 5 amino acids to about 100 amino acids, about 10 amino acids to about 100 amino acids, about 15 amino acids to about 100 amino acids, about 20 amino acids to about 100 amino acids, about 26 amino acids to about 100 amino acids, about 30 amino acids to about 100 amino acids, about 40 amino acids to about 100 amino acids, about 50 amino acids to about 100 amino acids, about 60 amino acids to about 100 amino acids, about 70 amino acids to about 100 amino acids, about 75 amino acids to about 100 amino acids, about 80 amino acids to about 100 amino acids, or about 90 amino acids to about 100 amino acids. For example, in some embodiments, the at least one epitope to which the first specific binding partner and/or the fifth specific binding partner binds is at least 5 amino acids in length, at least 6 amino acids in length, at least 7 amino acids in length, at least 8 amino acids in length, at least 9 amino acids in length, at least 10 amino acids in length, at least 11 amino acids in length, at least 12 amino acids in length, at least 13 amino acids in length, at least 14 amino acids in length, at least 15 amino acids in length, at least 16 amino acids in length, at least 17 amino acids in length, at least 18 amino acids in length, at least 19 amino acids in length, at least 20 amino acids in length, at least 21 amino acids in length, at least 22 amino acids in length, at least 23 amino acids in length, at least 24 amino acids in length, at least 25 amino acids in length, at least 26 amino acids in length, at least 27 amino acids in length, at least 28 amino acids in length, at least 29 amino acids in length or at least 30 amino acids in length. For example, the epitope from the SARS-CoV-2 spike protein or fragment or variant thereof against which the first specific binding partner and/or the fifth specific binding partner is directed can be derived from amino acids 319-542 of SEQ ID NO:3 or amino acids 306-528 of SEQ ID NO:4 or a fragment or variant thereof. A “fragment” of SEQ ID NO:3 or SEQ ID NO:4 refers to a protein or polypeptide that comprises a part that is less than the entirety of SEQ ID NO:3 or SEQ ID NO:4.

In some aspects, an isolated SARS-CoV-2 variant polypeptide can comprise one or more insertions, substitutions and/or deletions in one or more amino acid positions within SEQ ID NO:3 or SEQ ID NO:4. For example, an isolated SARS-CoV-2 variant polypeptide can comprise one or more insertions, substitutions and/or deletions in one or more amino acid positions within SEQ ID NO: 3 or SEQ ID NO:4 including those shown below in Table A1 and/or Table A2.

TABLE A1

| | | Strain | | | | | | | |
|---|---|---|---|---|---|---|---|---|---|
| | | Country | | | | | | | |
| | | Ohio | Ohio | | | | | | |
| | | COH.26G/ | COH.20G/ | Canada | Brazil | Brazil | USCF | UK | SA |
| Protein | Mutation | 677H | 501Y | L00311131 | P.1 | B.1.1.28 | UC301-3 | B.1.1.7 | 501Y.V2 |

| | | | | | | | | |
|---|---|---|---|---|---|---|---|---|
| Spike | N501Y | X | | X | | X | X | X |
| Spike | K417N | | | | | | | X |
| Spike | K417T | | | X | | | | |
| Spike | E484K | | | X | X | | | X |
| Spike | L452R | | | | | | | |
| Spike | S477N | | X | | | | | |
| Spike | A570D | | | | | | | |
| Spike | P681H | | | | | | | |
| Spike | T716I | | | | | | | |
| Spike | S982A | | | | | | | |
| Spike | D1118H | | | | | | | |

TABLE A2

Strain
Variant

| Protein | Mutation | Delta | Lambda | Omicron |
|---|---|---|---|---|
| Spike | T19R | X | | |
| Spike | A67V | | | X |
| Spike | H69 | | | Deletion |
| Spike | V70 | | | Deletion |
| Spike | G75V | | X | |
| Spike | T76I | | X | |
| Spike | T95I | | | X |
| Spike | G142 | | | Deletion |
| Spike | V143 | | | Deletion |
| Spike | Y144 | | | Deletion |
| Spike | Y145D | | | X |
| Spike | N211 | | | Deletion |
| Spike | L212I | | | X |
| Spike | 214EPE | | | insertion |
| Spike | A222V | X | | |
| Spike | R246A | | X | |
| Spike | S247A | | X | |
| Spike | Y248A | | X | |
| Spike | L249A | | X | |
| Spike | T250A | | X | |
| Spike | P251A | | X | |
| Spike | G252A | | X | |
| Spike | D253N | | X | |
| Spike | G339D | | | X |
| Spike | S371L | | | X |
| Spike | S373P | | | X |
| Spike | S375F | | | X |
| Spike | K417N | | | X |
| Spike | N440K | | | X |
| Spike | G446S | | | X |
| Spike | S477N | | | X |
| Spike | T478K | X | | X |
| Spike | E484A | | | X |
| Spike | F490S | | X | |
| Spike | Q493R | | | X |
| Spike | G496S | | | X |
| Spike | Q498R | | | X |
| Spike | N501Y | | | X |
| Spike | Y505H | | | X |
| Spike | T547K | | | X |
| Spike | L452R/Q | L452R | L452Q | |
| Spike | D614G | X | X | X |
| Spike | H655Y | | | X |
| Spike | N679K | | | |
| Spike | P681R/H | P681R | | PG81H |
| Spike | N764K | | | X |
| Spike | D796Y | | | X |
| Spike | N856K | | | X |
| Spike | T859N | | X | |
| Spike | D950N | X | | |
| Spike | Q954H | | | X |
| Spike | N969K | | | X |
| Spike | L981F | | | X |

In other aspects, the isolated SARS-CoV-2 variant polypeptide can comprise one or more insertions, substitutions and/or deletions in one or more of the following amino acid positions within SEQ ID NO:3 or SEQ ID NO:4: (1) replacing threonine with arginine at amino acid position 19 (T19R); (2) replacing alanine with valine at amino acid position 67 (A67V); (3) a deletion of histidine at amino acid position 69; (4) a deletion of valine at amino acid position 70; (5) replacing glycine with valine at amino acid position 75 (G75V); (6) replacing threonine with isoleucine at amino acid position 76 (T76I); (7) replacing threonine with isoleucine at amino acid position 85 (T95I); (8) a deletion of glycine at amino acid position 142; (9) a deletion of valine at amino acid position 143; (10) a deletion of tyrosine at amino acid position 144; (11) replacing a tyrosine with aspartic acid at amino acid 145 (Y145D); (12) a deletion of asparagine at amino acid 211; (13) replacing leucine with isoleucine at amino acid position 212 (L212I); (14) an insertion of glutamic acid, proline and glutamic acid (EPE) at amino acid position 214; (15) replacing alanine with valine at amino acid position 222 (A222V); (16) replacing arginine with alanine at amino acid position 246 (R246A); (17) replacing serine with alanine at amino acid position 247 (S247A); (18) replacing tyrosine with alanine at amino acid position 248 (Y248A); (19) replacing leucine with alanine at amino acid position 249 (L249A); (20) replacing threonine with alanine at amino acid position 250 (T250A); (21) replacing proline with alanine at amino acid position 251 (P251A); (22) replacing glycine with alanine at amino acid position 252 (G252A); (23) aspartic acid with asparagine at amino acid position 253 (D253N); (24) replacing glycine with aspartic acid at amino acid position 339 (G339D); (25) replacing serine with leucine at amino acid position 371 (S371L); (26) replacing serine with proline at amino acid position 373 (S373P); (27) replacing serine with phenylalanine at amino acid position 375 (S375F); (28) replacing lysine with asparagine or threonine at amino acid position 417 (K417N/T); (29) replacing asparagine with lysine at amino acid position 440 (N440K); (30) replacing glycine with serine at amino acid position 446 (G446S); (31) replacing leucine with arginine at amino acid position 452 (L452R); (32) replacing serine with asparagine at amino acid position 477 (S477N); (33) replacing threonine with lysine at amino acid position 478 (T478K); (34) replacing glutamic acid with alanine or lysine at amino acid position 484 (E484A/K); (35) replacing phenylalanine with serine at amino acid position 490 (F490S); (36) replacing glutamine with arginine at amino acid position 493 (Q493R); (37) replacing glycine with serine at amino acid position 496 (G496S); (38) replacing glutamine with arginine at amino acid position 498 (Q498R); (39) replacing asparagine with tyrosine at amino acid position 501 (N501Y); (40) replacing tyrosine with histidine at amino acid position 505 (Y505H); (41) replacing threonine with lysine at amino acid position 547 (T547K); (42) replacing lysine with arginine or glutamine at amino acid position 452 (L452R/Q); (43) replacing arginine with aspartic acid at amino acid position 570 (A570D); (44) replacing aspartic acid with glycine at amino acid position 614 (D614G); (45) replacing histidine with tyrosine at amino acid position 655 (H655Y); (46) replacing asparagine with lysine at amino acid position 679 (N679K); (47) replacing proline with arginine or histidine at amino acid position 681 (P681R/H); (48) replacing threonine with isoleucine at amino acid position 716 (T716I); (49) replacing asparagine with lysine at amino acid position 764 (N764K); (50) replacing aspartic acid with tyrosine at amino acid position 796 (D796Y); (51) replacing asparagine with lysine at amino acid position 856 (N856K); (52) replacing threonine with asparagine at amino acid position 859 (T859N); (53) replacing aspartic acid with asparagine at amino acid position 950 (D950N); (54) replacing glutamine with histidine at amino acid position 954 (Q954H); (55) replacing asparagine with lysine at amino acid position 969 (N969K); (56) replacing leucine with phenylalanine at amino acid position 981 (L981F); (57) replacing serine with alanine at amino acid position 982 (S982A); (58) replacing aspartic acid with histidine at amino acid position 1118 (D1118H); or (59) any combinations of (1)-(58) either alone or combined with any other substitutions and/or deletions within SEQ ID NO:3 or SEQ ID NO: 4.

Alternatively, the epitope can be derived from the RBD protein having the amino acid sequence of SEQ ID NOS:5 or 6 or a fragment or variant thereof. A “fragment” of SEQ ID NO:5 or SEQ ID NO:6 refers to a protein or polypeptide that comprises a part that is less than the entirety of SEQ ID NO:5 or SEQ ID NO:6.

In some embodiments, the at least one first specific binding partner and/or the at least one fifth specific binding partner comprise at least one anti-SARS-CoV-2 spike protein antibody, or antibody fragment or variant thereof, that specifically binds to at least one epitope within SARS-CoV-2 spike protein. The antibody or antibody fragment or variant thereof used as the at least one first specific binding partner and/or the at least one fifth specific binding partner is not critical and can be a polyclonal antibody, a monoclonal antibody, a humanized antibody, a chimeric antibody, a fully human antibody, a bispecific antibody, a multi-specific antibody, a single-chain variable fragment (“scFv”), a single chain antibody, a single domain antibody, a Fab fragment, a F(ab′) fragment, a F(ab′)₂ fragment, a disulfide-linked Fv (“sdFv”), or an anti-idiotypic (“anti-Id”) antibody, dual-domain antibody, dual variable domain (DVD) or triple variable domain (TVD) antibody. Examples of commercially antibodies that bind to an epitope on a SARS-CoV-2 spike protein and that can be used in the methods of the present disclosure include the antibodies provided in the SARS-CoV-2 (2019-nCoV) Spike ELISA Kit available from Sino Biological (Catalog Number KIT40591) or the COVID-19 Spike Protein ELISA Kit available from AbCam (Catalog Number ab274342). By way of another example, monoclonal antibody CR3022 can be used in the methods of the present disclosure. Specifically, monoclonal antibody CR3022 binds to an epitope on SARS-CoV and SARS-CoV-2 spike protein and is described in U.S. Pat. No. 8,106,170 (which describes the epitope), ter Meulen, et al., PLOS Medicine, 3(7):1071-1079 (July 2006) and Yuan et al., Science, published on-line on Apr. 3, 2020 (10.1126/science.abb7269) the contents of each of which are herein incorporated by reference.

Second and Sixth Specific Binding Partners. In some embodiments, the second specific binding partner and sixth specific binding partner are each specific for an epitope from the SARS-CoV-2 nucleocapsid protein or fragment or variant thereof. The nucleocapsid protein of at least one type of β-coronavirus (e.g., SARS-CoV and/or SARS-CoV-2) comprises two separate domains: a N-terminal domain (NTD) (also known as the N-terminal binding domain (NBD)) and a C-terminal domain (CTD) or (also known as the C-terminal binding domain (CBD). For example, the amino acid sequence of a nucleocapsid protein from a strain of human SARS-CoV-2 is set forth in SEQ ID NO: 1. The NTD can be found in amino acids 1-209 of SEQ ID NO:1. The CTD can be found in amino acids 210-419 of SEQ ID NO: 1. In some embodiments, the nucleocapsid protein to which the first and second specific binding members specifically binds comprises amino acids 1-209 of the nucleocapsid protein of at least one type of β-coronavirus, such as, for example, SARS-CoV or SARS-CoV-2, or any fragments or variants thereof. In yet another aspect, the nucleocapsid protein comprises amino acids 1-209 from a human SARS-CoV-2 (See, for example, SEQ ID NO:1).

In some embodiments, the second specific binding partner and sixth specific binding partner are each specific for an epitope in the N-terminal domain. In some embodiments, the second specific binding partner and sixth specific binding partner are each specific for an epitope in the C-terminal domain. In some embodiments, the second specific binding partner is specific for an epitope in the N-terminal domain and the sixth specific binding partner is specific for an epitope in the C-terminal domain. In some embodiments, the second specific binding partner is specific for an epitope in the C-terminal domain and the sixth specific binding partner is specific for an epitope in the N-terminal domain.

In another aspect, the epitope from the SARS-CoV-2 nucleocapsid protein or fragment or variant thereof against which the second specific binding partner and/or the sixth specific binding partner is directed can have a length of about 5 amino acids to about 500 amino acids, about 10 amino acids to about 500 amino acids, about 15 amino acids to about 500 amino acids, about 20 amino acids to about 500 amino acids, about 26 amino acids to about 500 amino acids, about 30 amino acids to about 500 amino acids, about 40 amino acids to about 500 amino acids, about 50 amino acids to about 500 amino acids, about 60 amino acids to about 500 amino acids, about 70 amino acids to about 500 amino acids, about 75 amino acids to about 500 amino acids, about 80 amino acids to about 500 amino acids, about 90 amino acids to about 500 amino acids, about 100 amino acids to about 500 amino acids, about 5 amino acids to about 400 amino acids, about 10 amino acids to about 400 amino acids, about 15 amino acids to about 400 amino acids, about 20 amino acids to about 400 amino acids, about 26 amino acids to about 400 amino acids, about 30 amino acids to about 400 amino acids, about 40 amino acids to about 400 amino acids, about 50 amino acids to about 400 amino acids, about 60 amino acids to about 400 amino acids, about 70 amino acids to about 400 amino acids, about 75 amino acids to about 400 amino acids, about 80 amino acids to about 400 amino acids, about 90 amino acids to about 400 amino acids, about 100 amino acids to about 400 amino acids, about 5 amino acids to about 300 amino acids, about 10 amino acids to about 300 amino acids, about 15 amino acids to about 300 amino acids, about 20 amino acids to about 300 amino acids, about 26 amino acids to about 300 amino acids, about 30 amino acids to about 300 amino acids, about 40 amino acids to about 300 amino acids, about 50 amino acids to about 300 amino acids, about 60 amino acids to about 300 amino acids, about 70 amino acids to about 300 amino acids, about 75 amino acids to about 300 amino acids, about 80 amino acids to about 300 amino acids, about 90 amino acids to about 300 amino acids, about 100 amino acids to about 300 amino acids, about 5 amino acids to about 200 amino acids, about 10 amino acids to about 200 amino acids, about 15 amino acids to about 200 amino acids, about 20 amino acids to about 200 amino acids, about 26 amino acids to about 200 amino acids, about 30 amino acids to about 200 amino acids, about 40 amino acids to about 200 amino acids, about 50 amino acids to about 200 amino acids, about 60 amino acids to about 200 amino acids, about 70 amino acids to about 200 amino acids, about 75 amino acids to about 200 amino acids, about 80 amino acids to about 200 amino acids, about 90 amino acids to about 200 amino acids, about 100 amino acids to about 200 amino acids, about 5 amino acids to about 100 amino acids, about 10 amino acids to about 100 amino acids, about 15 amino acids to about 100 amino acids, about 20 amino acids to about 100 amino acids, about 26 amino acids to about 100 amino acids, about 30 amino acids to about 100 amino acids, about 40 amino acids to about 100 amino acids, about 50 amino acids to about 100 amino acids, about 60 amino acids to about 100 amino acids, about 70 amino acids to about 100 amino acids, about 75 amino acids to about 100 amino acids, about 80 amino acids to about 100 amino acids, or about 90 amino acids to about 100 amino acids. For example, in some embodiments, the at least one epitope to which the second specific binding partner and/or the sixth specific binding partner binds is at least 5 amino acids in length, at least 6 amino acids in length, at least 7 amino acids in length, at least 8 amino acids in length, at least 9 amino acids in length, at least 10 amino acids in length, at least 11 amino acids in length, at least 12 amino acids in length, at least 13 amino acids in length, at least 14 amino acids in length, at least 15 amino acids in length, at least 16 amino acids in length, at least 17 amino acids in length, at least 18 amino acids in length, at least 19 amino acids in length, at least 20 amino acids in length, at least 21 amino acids in length, at least 22 amino acids in length, at least 23 amino acids in length, at least 24 amino acids in length, at least 25 amino acids in length, at least 26 amino acids in length, at least 27 amino acids in length, at least 28 amino acids in length, at least 29 amino acids in length or at least 30 amino acids in length. For example, the epitope from the SARS-CoV-2 nucleocapsid protein or fragment or variant thereof against which the second specific binding partner and/or the sixth specific binding partner is directed can be derived from an amino acid sequence of SEQ ID NOS:1 or 2.

In some embodiments, the second specific binding partner and sixth specific binding partner each comprise at least one anti-SARS-CoV-2 nucleocapsid protein antibody, or antibody fragment or variant thereof, that specifically binds to at least one epitope within SARS-CoV-2 nucleocapsid protein. The antibody or antibody fragment or variant thereof used as the second specific binding partner and/or the sixth specific binding partner is not critical and can be a polyclonal antibody, a monoclonal antibody, a humanized antibody, a chimeric antibody, a fully human antibody, a bispecific antibody, a multi-specific antibody, a single-chain variable fragment (“scFv”), a single chain antibody, a single domain antibody, a Fab fragment, a F(ab′) fragment, a F(ab′)₂ fragment, a disulfide-linked Fv (“sdFv”), or an anti-idiotypic (“anti-Id”) antibody, dual-domain antibody, dual variable domain (DVD) or triple variable domain (TVD) antibody. For example, in some aspects, (1) the second specific binding partner comprises an anti-SARS-CoV-2 antibody or antibody fragment thereof which specifically binds to at least one epitope within amino acids 110 to 210 of a N-terminus of at least one SARS-CoV-2 nucleocapsid antigen (e.g., SEQ ID NO:1) in the sample; and (2) the sixth specific binding partner comprises at least one detectable label that specifically binds to at least one epitope within amino acids 82-101 of a N-terminus of the at least one SARS-CoV-2 nucleocapsid antigen (e.g., SEQ ID NO:1). In some aspects, the sixth specific binding partner comprises at least one detectable label that specifically binds to at least one epitope within amino acids 82-10 of the N-terminus of the at least one SARS-CoV-2 nucleocapsid antigen (e.g., SEQ ID NO:1). More specifically, an example of an antibody that binds to amino acids 82-101 of the N-terminus of SARS-CoV-2 that can be used in the methods of the present disclosure is described in Tsunetsugu-Yokota et al., Rev. Med. Virol., 16: 117-131 (2006), the contents of which are herein incorporated by reference. Alternatively, other antibodies or antibody fragments or variants thereof that specific for an epitope from the SARS-CoV-2 nucleocapsid protein or fragment or variant thereof that can be used in the methods of the present disclosure include those antibodies in the Sampinute COVID-19 Antigen MIA test available from Celltrion USA, Inc., the BD Veritor System for Rapid Detection of SARS-CoV-2 test available from Becton, Dickinson and Company, BinaxNOW COVID-19 Antigen Card available from Abbott Diagnostics Scarborough Inc., and the QuickVue SARS Antigen Test available from Quidel Corporation.

Third and Seventh Specific Binding Partners. In some embodiments, the at least one third specific binding partner and the at least one seventh specific binding partner comprises at least one recombinant SARS-CoV-2 spike protein antigen or fragment or variant thereof. In some embodiments, the recombinant antigen or fragment or variant thereof of the at least one third specific binding partner and/or the at least on seventh specific binding partner comprises all or at least a portion of at least one SARS-CoV-2 isolated polypeptide or fragment or variant thereof from a spike protein or variant thereof. As described above, the spike protein comprises S1 and S2 polypeptides. The S1 polypeptide contains the receptor binding domain (RBD) of the protein, while the S2 polypeptide forms the stalk of the spike molecule.

Any spike protein from SARS-CoV-2, known in the art can be used in the at least one third specific binding partner and the at least one seventh specific binding partner. For example, spike proteins from SARS-CoV-2 such as those described, for example, in Lu et al., Lancet, 395:565-574 (February 2020) and deposited in the China National Microbiological Data Center (Accession number NMDC10013002 and Genome accession numbers NMDC60013002-01 to NMDC60013002-10), Wuhan-Hu-1 (GenBank Accession No. NC_045512.2), Wuhan-Hu-1 (GenBank Accession No. MN908947.3) and www.ncbi.nlm.nih.gov/genbank/sars-cov-2-seqs/, the contents of which are herein incorporated by reference, can be used.

In some embodiments, the spike protein or fragment or variant thereof of the at least one third specific binding partner and/or the at least on seventh specific binding partner can have a length of about 5 to about 1300 amino acids (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 272, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1272, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, or 1300 amino acids).

In another aspect, the spike protein or fragment or variant thereof of the at least one third specific binding partner and/or the at least on seventh specific binding partner can have a length of about 5 amino acids to about 1500 amino acids, about 10 amino acids to about 1500 amino acids, about 15 amino acids to about 1500 amino acids, about 20 amino acids to about 1500 amino acids, about 25 amino acids to about 1500 amino acids, about 30 amino acids to about 1500 amino acids, about 40 amino acids to about 1500 amino acids, about 50 amino acids to about 1500 amino acids, about 60 amino acids to about 1500 amino acids, about 70 amino acids to about 1500 amino acids, about 75 amino acids to about 1500 amino acids, about 80 amino acids to about 1500 amino acids, about 90 amino acids to about 1500 amino acids, about 100 amino acids to about 1500 amino acids, about 5 amino acids to about 1400 amino acids, about 10 amino acids to about 1400 amino acids, about 15 amino acids to about 1400 amino acids, about 20 amino acids to about 1400 amino acids, about 25 amino acids to about 1400 amino acids, about 30 amino acids to about 1400 amino acids, about 40 amino acids to about 1400 amino acids, about 50 amino acids to about 1400 amino acids, about 60 amino acids to about 1400 amino acids, about 70 amino acids to about 1400 amino acids, about 75 amino acids to about 1400 amino acids, about 80 amino acids to about 1400 amino acids, about 90 amino acids to about 1400 amino acids, about 100 amino acids to about 1400 amino acids, about 5 amino acids to about 1300 amino acids, about 10 amino acids to about 1300 amino acids, about 15 amino acids to about 1300 amino acids, about 20 amino acids to about 1300 amino acids, about 25 amino acids to about 1300 amino acids, about 30 amino acids to about 1300 amino acids, about 40 amino acids to about 1300 amino acids, about 50 amino acids to about 1300 amino acids, about 60 amino acids to about 1300 amino acids, about 70 amino acids to about 1300 amino acids, about 75 amino acids to about 1300 amino acids, about 80 amino acids to about 1300 amino acids, about 90 amino acids to about 1300 amino acids, about 100 amino acids to about 1300 amino acids, about 5 amino acids to about 1200 amino acids, about 10 amino acids to about 1200 amino acids, about 15 amino acids to about 1200 amino acids, about 20 amino acids to about 1200 amino acids, about 25 amino acids to about 1200 amino acids, about 30 amino acids to about 1200 amino acids, about 40 amino acids to about 1200 amino acids, about 50 amino acids to about 1200 amino acids, about 60 amino acids to about 1200 amino acids, about 70 amino acids to about 1200 amino acids, about 75 amino acids to about 1020 amino acids, about 80 amino acids to about 1200 amino acids, about 90 amino acids to about 1200 amino acids, about 100 amino acids to about 1200 amino acids, about 5 amino acids to about 1100 amino acids, about 10 amino acids to about 1100 amino acids, about 15 amino acids to about 1100 amino acids, about 20 amino acids to about 1100 amino acids, about 25 amino acids to about 1100 amino acids, about 30 amino acids to about 1100 amino acids, about 40 amino acids to about 1100 amino acids, about 50 amino acids to about 1100 amino acids, about 60 amino acids to about 1100 amino acids, about 70 amino acids to about 1100 amino acids, about 75 amino acids to about 1100 amino acids, about 80 amino acids to about 1100 amino acids, about 90 amino acids to about 1100 amino acids, about 100 amino acids to about 1100 amino acids, about 5 amino acids to about 1000 amino acids, about 10 amino acids to about 1000 amino acids, about 15 amino acids to about 1000 amino acids, about 20 amino acids to about 1000 amino acids, about 25 amino acids to about 1000 amino acids, about 30 amino acids to about 1000 amino acids, about 40 amino acids to about 1000 amino acids, about 50 amino acids to about 1000 amino acids, about 60 amino acids to about 1000 amino acids, about 70 amino acids to about 1000 amino acids, about 75 amino acids to about 1000 amino acids, about 80 amino acids to about 1000 amino acids, about 90 amino acids to about 1000 amino acids, about 100 amino acids to about 1000 amino acids, about 5 amino acids to about 900 amino acids, about 10 amino acids to about 900 amino acids, about 15 amino acids to about 900 amino acids, about 20 amino acids to about 900 amino acids, about 25 amino acids to about 900 amino acids, about 30 amino acids to about 900 amino acids, about 40 amino acids to about 900 amino acids, about 50 amino acids to about 900 amino acids, about 60 amino acids to about 900 amino acids, about 70 amino acids to about 900 amino acids, about 75 amino acids to about 900 amino acids, about 80 amino acids to about 900 amino acids, about 90 amino acids to about 900 amino acids, about 100 amino acids to about 900 amino acids, about 5 amino acids to about 800 amino acids, about 10 amino acids to about 800 amino acids, about 15 amino acids to about 800 amino acids, about 20 amino acids to about 800 amino acids, about 25 amino acids to about 800 amino acids, about 30 amino acids to about 800 amino acids, about 40 amino acids to about 800 amino acids, about 50 amino acids to about 800 amino acids, about 60 amino acids to about 800 amino acids, about 70 amino acids to about 800 amino acids, about 75 amino acids to about 800 amino acids, about 80 amino acids to about 800 amino acids, about 90 amino acids to about 800 amino acids, about 100 amino acids to about 800 amino acids, about 5 amino acids to about 700 amino acids, about 10 amino acids to about 700 amino acids, about 15 amino acids to about 700 amino acids, about 20 amino acids to about 700 amino acids, about 25 amino acids to about 700 amino acids, about 30 amino acids to about 700 amino acids, about 40 amino acids to about 700 amino acids, about 50 amino acids to about 700 amino acids, about 60 amino acids to about 700 amino acids, about 70 amino acids to about 700 amino acids, about 75 amino acids to about 700 amino acids, about 80 amino acids to about 700 amino acids, about 90 amino acids to about 700 amino acids, about 100 amino acids to about 700 amino acids, about 5 amino acids to about 600 amino acids, about 10 amino acids to about 600 amino acids, about 15 amino acids to about 600 amino acids, about 20 amino acids to about 600 amino acids, about 25 amino acids to about 600 amino acids, about 30 amino acids to about 600 amino acids, about 40 amino acids to about 600 amino acids, about 50 amino acids to about 600 amino acids, about 60 amino acids to about 600 amino acids, about 70 amino acids to about 600 amino acids, about 75 amino acids to about 600 amino acids, about 80 amino acids to about 600 amino acids, about 90 amino acids to about 600 amino acids, about 100 amino acids to about 600 amino acids, about 5 amino acids to about 500 amino acids, about 10 amino acids to about 500 amino acids, about 15 amino acids to about 500 amino acids, about 20 amino acids to about 500 amino acids, about 25 amino acids to about 500 amino acids, about 30 amino acids to about 500 amino acids, about 40 amino acids to about 500 amino acids, about 50 amino acids to about 500 amino acids, about 60 amino acids to about 500 amino acids, about 70 amino acids to about 500 amino acids, about 75 amino acids to about 500 amino acids, about 80 amino acids to about 500 amino acids, about 90 amino acids to about 500 amino acids, about 100 amino acids to about 500 amino acids, about 5 amino acids to about 400 amino acids, about 10 amino acids to about 400 amino acids, about 15 amino acids to about 400 amino acids, about 20 amino acids to about 400 amino acids, about 25 amino acids to about 400 amino acids, about 30 amino acids to about 400 amino acids, about 40 amino acids to about 400 amino acids, about 50 amino acids to about 400 amino acids, about 60 amino acids to about 400 amino acids, about 70 amino acids to about 400 amino acids, about 75 amino acids to about 400 amino acids, about 80 amino acids to about 400 amino acids, about 90 amino acids to about 400 amino acids, about 100 amino acids to about 400 amino acids, about 5 amino acids to about 300 amino acids, about 10 amino acids to about 300 amino acids, about 15 amino acids to about 300 amino acids, about 20 amino acids to about 300 amino acids, about 25 amino acids to about 300 amino acids, about 30 amino acids to about 300 amino acids, about 40 amino acids to about 300 amino acids, about 50 amino acids to about 300 amino acids, about 60 amino acids to about 300 amino acids, about 70 amino acids to about 300 amino acids, about 75 amino acids to about 300 amino acids, about 80 amino acids to about 300 amino acids, about 90 amino acids to about 300 amino acids, about 100 amino acids to about 300 amino acids, about 5 amino acids to about 200 amino acids, about 10 amino acids to about 200 amino acids, about 15 amino acids to about 200 amino acids, about 20 amino acids to about 200 amino acids, about 25 amino acids to about 200 amino acids, about 30 amino acids to about 200 amino acids, about 40 amino acids to about 200 amino acids, about 50 amino acids to about 200 amino acids, about 60 amino acids to about 200 amino acids, about 70 amino acids to about 200 amino acids, about 75 amino acids to about 200 amino acids, about 80 amino acids to about 200 amino acids, about 90 amino acids to about 200 amino acids, about 100 amino acids to about 200 amino acids, about 5 amino acids to about 100 amino acids, about 10 amino acids to about 100 amino acids, about 15 amino acids to about 100 amino acids, about 20 amino acids to about 100 amino acids, about 25 amino acids to about 100 amino acids, about 30 amino acids to about 100 amino acids, about 40 amino acids to about 100 amino acids, about 50 amino acids to about 100 amino acids, about 60 amino acids to about 100 amino acids, about 70 amino acids to about 100 amino acids, about 75 amino acids to about 100 amino acids, about 80 amino acids to about 100 amino acids, or about 90 amino acids to about 100 amino acids.

In some embodiments, the spike protein or fragment or variant thereof of the at least one third specific binding partner and/or the at least on seventh specific binding partner comprises the RBD of a spike protein of SARS-CoV-2 or any fragments or variants thereof.

In other aspect, the spike protein comprises amino acids 319-542 of SEQ ID NO:3 or amino acids 306-528 of SEQ ID NO:4 or a fragment or variant thereof. A “fragment” of SEQ ID NO:3 or SEQ ID NO:4 refers to a protein or polypeptide that comprises a part that is less than the entirety of SEQ ID NO:3 or SEQ ID NO:4.

In yet another aspect, the RBD protein has the sequence of RVQPTESIVRFPNITNLCPFGEVFNATRFASVYAWNRKRISNCVADYSVLYNSASFSTFK CYGVSPTKLNDLCFTNVYADSFVIRGDEVRQIAPGQTGKIADYNYKLPDDFTGCVIAWN SNNLDSKVGGNYNYLYRLFRKSNLKPFERDISTEIYQAGSTPCNGVEGFNCYFPLQSYGF QPTNGVGYQPYRVVVLSFELLHAPATVCGPKKSTNLVKNKCVNFXaa (SEQ ID NO:5) or a fragment or variant thereof, wherein Xₐₐ, is absent or present, and, if present, is an N or S. A “fragment” of SEQ ID NO:5 refers to a protein or polypeptide that comprises a part that is less than the entirety of SEQ ID NO:5. A fragment of SEQ ID NO:5 can comprise from about 5 to about 200 contiguous amino acids. In another aspect, a fragment of SEQ ID NO:5 comprises at least about 5 contiguous amino acids of SEQ ID NO:5, at least about 10 contiguous amino acids of SEQ ID NO:5, at least about 15 contiguous amino acids of SEQ ID NO:5, at least about 20 contiguous amino acids of SEQ ID NO:5, at least about 25 contiguous amino acids of SEQ ID NO:5, at least about 30 contiguous amino acids of SEQ ID NO:5, at least about 35 contiguous amino acids of SEQ ID NO:5, at least about 40 contiguous amino acids of SEQ ID NO:5, at least about 45 contiguous amino acids of SEQ ID NO:5, at least about 50 contiguous amino acids of SEQ ID NO:5, at least about 55 contiguous amino acids of SEQ ID NO:5, at least about 60 contiguous amino acids of SEQ ID NO:5, at least about 65 contiguous amino acids of SEQ ID NO:5, at least about 70 contiguous amino acids of SEQ ID NO:5, at least about 75 contiguous amino acids of SEQ ID NO:5, at least about 80 contiguous amino acids of SEQ ID NO:5, at least about 85 contiguous amino acids of SEQ ID NO:5, at least about 90 contiguous amino acids of SEQ ID NO:5, at least about 95 contiguous amino acids of SEQ ID NO:5, at least 100 contiguous amino acids of SEQ ID NO:5, at least about 105 contiguous amino acids of SEQ ID NO:5, at least about 110 contiguous amino acids of SEQ ID NO:5, at least about 115 contiguous amino acids of SEQ ID NO:5, at least about 120 contiguous amino acids of SEQ ID NO:5, at least about 125 contiguous amino acids of SEQ ID NO:5, at least about 130 contiguous amino acids of SEQ ID NO:5, at least about 135 contiguous amino acids of SEQ ID NO:5, at least about 140 contiguous amino acids of SEQ ID NO:5, at least about 145 contiguous amino acids of SEQ ID NO:5, at least about 150 contiguous amino acids of SEQ ID NO:5, at least about 55 contiguous amino acids of SEQ ID NO:5, at least about 160 contiguous amino acids of SEQ ID NO:5, at least about 165 contiguous amino acids of SEQ ID NO:5, at least about 170 contiguous amino acids of SEQ ID NO:5, at least about 175 contiguous amino acids of SEQ ID NO:5, at least about 180 contiguous amino acids of SEQ ID NO:5, at least about 85 contiguous amino acids of SEQ ID NO:5, at least about 190 contiguous amino acids of SEQ ID NO:5, at least about 95 contiguous amino acids of SEQ ID NO:5, at least 200 contiguous amino acids of SEQ ID NO:5 or at least about 205 contiguous amino acids of SEQ ID NO:5.

In yet another aspect, the RBD protein has the sequence of RVVPSGDVVRFPNITNLCPFGEVFNATKFPSVYAWERKKISNCVADYSVLYNSTF FSTFKCYGVSATKLNDLCFSNVYADSFVVKGDDVRQIAPGQTGVIADYNYKLPDDFMG CVLAWNTRNIDATSTGNYNYKYRYLRHGKLRPFERDISNVPFSPDGKPCTPPALNCYWP LNDYGFYTTTGIGYQPYRVVVLSFELLNAPATVCGPKLSTDLIKNQCVNF Xₐₐ (SEQ ID NO:6) or a fragment or variant thereof, wherein Xₐₐ is either absent or present, and, if present, is a N or S. A “fragment” of SEQ ID NO:6 refers to a protein or polypeptide that comprises a part that is less than the entirety of SEQ ID NO:6. A fragment of SEQ ID NO:6 can comprise from about 5 to about 200 contiguous amino acids. In another aspect, a fragment of SEQ ID NO:6 comprises at least about 5 contiguous amino acids of SEQ ID NO:6, at least about 10 contiguous amino acids of SEQ ID NO:6, at least about 15 contiguous amino acids of SEQ ID NO:6, at least about 20 contiguous amino acids of SEQ ID NO:6, at least about 25 contiguous amino acids of SEQ ID NO:6, at least about 30 contiguous amino acids of SEQ ID NO:6, at least about 35 contiguous amino acids of SEQ ID NO:6, at least about 40 contiguous amino acids of SEQ ID NO:6, at least about 45 contiguous amino acids of SEQ ID NO:6, at least about 50 contiguous amino acids of SEQ ID NO:6, at least about 55 contiguous amino acids of SEQ ID NO:6, at least about 60 contiguous amino acids of SEQ ID NO:6, at least about 65 contiguous amino acids of SEQ ID NO:6, at least about 70 contiguous amino acids of SEQ ID NO:6, at least about 75 contiguous amino acids of SEQ ID NO:6, at least about 80 contiguous amino acids of SEQ ID NO:6, at least about 85 contiguous amino acids of SEQ ID NO:6, at least about 90 contiguous amino acids of SEQ ID NO:6, at least about 95 contiguous amino acids of SEQ ID NO:6, at least 100 contiguous amino acids of SEQ ID NO:6, at least about 105 contiguous amino acids of SEQ ID NO:6, at least about 110 contiguous amino acids of SEQ ID NO:6, at least about 115 contiguous amino acids of SEQ ID NO:6, at least about 120 contiguous amino acids of SEQ ID NO:6, at least about 125 contiguous amino acids of SEQ ID NO:6, at least about 130 contiguous amino acids of SEQ ID NO:6, at least about 135 contiguous amino acids of SEQ ID NO:6, at least about 140 contiguous amino acids of SEQ ID NO:6, at least about 145 contiguous amino acids of SEQ ID NO:6, at least about 150 contiguous amino acids of SEQ ID NO:6, at least about 55 contiguous amino acids of SEQ ID NO:6, at least about 160 contiguous amino acids of SEQ ID NO:6, at least about 165 contiguous amino acids of SEQ ID NO:6, at least about 180 contiguous amino acids of SEQ ID NO:6, at least about 185 contiguous amino acids of SEQ ID NO:6, at least about 180 contiguous amino acids of SEQ ID NO:6, at least about 85 contiguous amino acids of SEQ ID NO:6, at least about 190 contiguous amino acids of SEQ ID NO:6, at least about 95 contiguous amino acids of SEQ ID NO:6, at least 200 contiguous amino acids of SEQ ID NO:6 or at least about 205 contiguous amino acids of SEQ ID NO:6.

In some embodiments, the recombinant antigen of the at least one third specific binding partner and/or the at least on seventh specific binding partner comprises is a fusion protein comprising at least all or at least a portion of SARS-CoV-2 spike protein or fragment or variant thereof. In another aspect, the fusion protein may comprise all or at least portion (e.g., at least 5 amino acids or more) of the S1 polypeptide, S2 polypeptide, and/or RBD of a spike protein from SARS-CoV-2 operably linked, fused or grafted directly or indirectly (such as through one or more linking peptide sequences and/or HIS tags) to another protein or polypeptide.

Fourth and Eighth Specific Binding Partners. In some embodiments, the at least one fourth specific binding partner and the at least one eighth specific binding partner comprise at least one recombinant SARS-CoV-2 nucleocapsid protein antigen or fragment or variant thereof. In some embodiments, the at least one fourth specific binding partner and the at least one eighth specific binding partner comprises at least one recombinant SARS-CoV-2 nucleocapsid protein, or fragment or variant thereof.

Any nucleocapsid protein from SARS-CoV-2 known in the art can be used in the at least one first specific binding partner. For example, nucleocapsid proteins from SARS-CoV-2 such as those described, for example, in Lu et al., Lancet, 395:565-574 (February 2020) and deposited in the China National Microbiological Data Center (Accession number NMDC10013002 and Genome accession numbers NMDC60013002-01 to NMDC60013002-10), Wuhan-Hu-1 (GenBank Accession No. NC_045512.2), Wuhan-Hu-1 (GenBank Accession No. MN908947.3) and www.ncbi.nlm.nih.gov/genbank/sars-cov-2-seqs/, the contents of which are herein incorporated by reference, can be used.

The nucleocapsid protein, fragment or variant thereof of the at least one fourth specific binding partner and the at least one eighth specific binding partner can have a length of about 5 to about 500 amino acids (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 272, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids).

In another aspect, the nucleocapsid protein, fragment or variant thereof of the at least one fourth specific binding partner and the at least one eighth specific binding partner can have a length of about 5 amino acids to about 500 amino acids, about 10 amino acids to about 500 amino acids, about 15 amino acids to about 500 amino acids, about 20 amino acids to about 500 amino acids, about 25 amino acids to about 500 amino acids, about 30 amino acids to about 500 amino acids, about 40 amino acids to about 500 amino acids, about 50 amino acids to about 500 amino acids, about 60 amino acids to about 500 amino acids, about 70 amino acids to about 500 amino acids, about 75 amino acids to about 500 amino acids, about 80 amino acids to about 500 amino acids, about 90 amino acids to about 500 amino acids, about 100 amino acids to about 500 amino acids, about 5 amino acids to about 400 amino acids, about 10 amino acids to about 400 amino acids, about 15 amino acids to about 400 amino acids, about 20 amino acids to about 400 amino acids, about 25 amino acids to about 400 amino acids, about 30 amino acids to about 400 amino acids, about 40 amino acids to about 400 amino acids, about 50 amino acids to about 400 amino acids, about 60 amino acids to about 400 amino acids, about 70 amino acids to about 400 amino acids, about 75 amino acids to about 400 amino acids, about 80 amino acids to about 400 amino acids, about 90 amino acids to about 400 amino acids, about 100 amino acids to about 400 amino acids, about 5 amino acids to about 300 amino acids, about 10 amino acids to about 300 amino acids, about 15 amino acids to about 300 amino acids, about 20 amino acids to about 300 amino acids, about 25 amino acids to about 300 amino acids, about 30 amino acids to about 300 amino acids, about 40 amino acids to about 300 amino acids, about 50 amino acids to about 300 amino acids, about 60 amino acids to about 300 amino acids, about 70 amino acids to about 300 amino acids, about 75 amino acids to about 300 amino acids, about 80 amino acids to about 300 amino acids, about 90 amino acids to about 300 amino acids, about 100 amino acids to about 300 amino acids, about 5 amino acids to about 200 amino acids, about 10 amino acids to about 200 amino acids, about 15 amino acids to about 200 amino acids, about 20 amino acids to about 200 amino acids, about 25 amino acids to about 200 amino acids, about 30 amino acids to about 200 amino acids, about 40 amino acids to about 200 amino acids, about 50 amino acids to about 200 amino acids, about 60 amino acids to about 200 amino acids, about 70 amino acids to about 200 amino acids, about 75 amino acids to about 200 amino acids, about 80 amino acids to about 200 amino acids, about 90 amino acids to about 200 amino acids, about 100 amino acids to about 200 amino acids, about 5 amino acids to about 100 amino acids, about 10 amino acids to about 100 amino acids, about 15 amino acids to about 100 amino acids, about 20 amino acids to about 100 amino acids, about 25 amino acids to about 100 amino acids, about 30 amino acids to about 100 amino acids, about 40 amino acids to about 100 amino acids, about 50 amino acids to about 100 amino acids, about 60 amino acids to about 100 amino acids, about 70 amino acids to about 100 amino acids, about 75 amino acids to about 100 amino acids, about 80 amino acids to about 100 amino acids, or about 90 amino acids to about 100 amino acids.

In some embodiments, the nucleocapsid protein, fragment or variant thereof of the at least one fourth specific binding partner and the at least one eighth specific binding partner comprises the CTD of a nucleocapsid protein of SARS-CoV-2 or any fragments or variants thereof.

In other embodiments, the at least one fourth specific binding partner and/or the at least one eighth specific binding partner comprises amino acids 210-419 of the nucleocapsid protein of at least one type of β-coronavirus, such as, for example, SARS-CoV or SARS-CoV-2, or any fragments or variants thereof. In yet another aspect, the nucleocapsid protein comprises amino acids 210-419 from a human SARS-CoV-2 (See, for example, SEQ ID NO:2) or a fragment or variant thereof. A “fragment” of SEQ ID NO:2 refers to a protein or polypeptide that comprises a part that is less than the entirety of SEQ ID NO:2.

In some aspects, an isolated SARS-CoV-2 variant polypeptide can comprise one or more insertions, substitutions and/or deletions in one or more amino acid positions within SEQ ID NO:2. For example, an isolated SARS-CoV-2 variant polypeptide can comprise one or more insertions, substitutions and/or deletions in one or more amino acid positions within SEQ ID NO:2 including those shown below in Tables B1 and/or B2.

TABLE B1

| | | Strain | | | | | | | |
|---|---|---|---|---|---|---|---|---|---|
| | | Country | | | | | | | |
| | | Ohio | Ohio | | | | | | |
| | | COH.26G/ | COH.20G/ | Canada | Brazil | Brazil | USCF | UK | SA |
| Protein | Mutation | 677H | 501Y | L00311131 | P.1 | B.1.1.28 | UC301-3 | B.1.1.7 | 501Y.V2 |
| Nucleocapsid | D3L | | | | | | | | |
| Nucleocapsid | S235F | | | | | | X | X | |
| Nucleocapsid | M234I | | | X | | X | | | |
| Nucleocapsid | K373N | | | | | | X | | |
| Nucleocapsid | D377Y | X | | | | | | | |
| Nucleocapsid | A376T | | | X | | | | | |

TABLE B2

Strain
Variant

| Protein | Mutation | Delta | Lambda | Omicron |
|---|---|---|---|---|
| Nucleocapsid | P13L | | X | |
| Nucleocapsid | D63G | X | | |
| Nucleocapsid | R203M/K | R203M | R203K | R203K |
| Nucleocapsid | G204R | | X | X |
| Nucleocapsid | G214C | | X | |
| 1Nucleocapsid | Q349H | X | | |
| Nucleocapsid | D377Y | X | | |
| Nucleocapsid | P432L | | X | |
| Nucleocapsid | R622K | | X | |
| Nucleocapsid | G623R | | X | |
| Nucleocapsid | G633C | | X | |

In some other aspects, the isolated SARS-CoV-2 variant polypeptide can comprise one or more insertions, substitutions and/or deletions in one or more positions within SEQ ID NO:2 and/or one or more insertions, substitutions and/or deletions in one or more amino acid positions within SEQ ID NO:3 or SEQ ID NO:4. For example, the isolated SARS-CoV-2 variant polypeptide can comprise one or more insertions, substitutions and/or deletions in one or more of the amino acid positions in SEQ ID NO:2 including those shown in Table B1 and/or B2 and/or one or more insertions, substitutions and/or deletions in one or more of the amino acid positions within SEQ ID NO:3 or SEQ ID NO:4 as shown in Tables A1 and/or A2.

In other aspects, the isolated SARS-CoV-2 variant polypeptide can comprise one or more insertions, substitutions and/or deletions in one or more of the following amino acid positions within SEQ ID NO:2: (1) replacing aspartic acid with leucine at amino acid position 3 (D3L); (2) proline with leucine at amino acid position 13 (P13L); (3) replacing aspartic acid with glycine at amino acid position 63 (D63G); (4) replacing arginine with methionine or lysine at amino acid position 302 (R203M/K); (5) replacing glycine with arginine at amino acid position 204 (G204R); (6) replacing glycine with cysteine at amino acid position 214 (G214C); (7) replacing serine with phenylalanine at amino acid position 235 (S235F); (8) replacing methionine with isoleucine at amino acid position 234 (M234I); (9) replacing glutamine with histidine at amino acid position 349 (Q349H); (10) replacing lysine with asparagine at amino acid position 373 (K373N); (11) replacing alanine with threonine at amino acid position 376 (A376T); (12) replacing aspartic acid with tyrosine at amino acid position 377 (D377Y); (13) replacing proline with leucine at amino acid position 432 (P432L); (14) replacing arginine with lysine at amino acid position 622 (R622K); (15) replacing glycine with arginine at amino acid position 623 (G623R); (16) replacing glycine with cysteine at amino acid position 633 (G633C); or (17) any combinations of (1)-(16), either alone or combined with any other substitutions and/or deletions within SEQ ID NO:2.

In still yet another aspect, the nucleocapsid protein has the sequence of: MAGNGGDAALALLLLDRLNQLESKMSGKGQQQQGQTVTKKSAAEASKKPRQKRTAT KAYNVTQAFGRRGPEQTQGNFGDQELIRQGTDYKHWPQIAQFAPSASAFFGMSRIGME VTPSGTWLTYTGAIKLDDKDPNFKDQVILLNKHIDAYKTFPPTEPKKDKKKKADETQAL PQRQKKQQTVTLLPAADLDDFSKQLQQSMSSADSTQA (SEQ ID NO:1) or fragment or variant thereof. A “fragment” of SEQ ID NO:1 refers to a protein or polypeptide that comprises a part that is less than the entirety of SEQ ID NO:1. A fragment of SEQ ID NO:1 can comprise from about 5 to about 200 contiguous amino acids. In another aspect, a fragment of SEQ ID NO:1 comprises at least about 5 contiguous amino acids of SEQ ID NO:1, at least about 10 contiguous amino acids of SEQ ID NO:1, at least about 15 contiguous amino acids of SEQ ID NO:1, at least about 20 contiguous amino acids of SEQ ID NO:1, at least about 25 contiguous amino acids of SEQ ID NO:1, at least about 30 contiguous amino acids of SEQ ID NO:1, at least about 35 contiguous amino acids of SEQ ID NO:1, at least about 40 contiguous amino acids of SEQ ID NO:1, at least about 45 contiguous amino acids of SEQ ID NO:1, at least about 50 contiguous amino acids of SEQ ID NO:1, at least about 55 contiguous amino acids of SEQ ID NO:1, at least about 60 contiguous amino acids of SEQ ID NO:1, at least about 65 contiguous amino acids of SEQ ID NO:1, at least about 70 contiguous amino acids of SEQ ID NO:1, at least about 75 contiguous amino acids of SEQ ID NO:1, at least about 80 contiguous amino acids of SEQ ID NO:1, at least about 85 contiguous amino acids of SEQ ID NO:1, at least about 90 contiguous amino acids of SEQ ID NO:1, at least about 95 contiguous amino acids of SEQ ID NO:1, at least 100 contiguous amino acids of SEQ ID NO:1, at least about 105 contiguous amino acids of SEQ ID NO:1, at least about 110 contiguous amino acids of SEQ ID NO:1, at least about 115 contiguous amino acids of SEQ ID NO: 1, at least about 120 contiguous amino acids of SEQ ID NO:1, at least about 125 contiguous amino acids of SEQ ID NO:1, at least about 130 contiguous amino acids of SEQ ID NO:1, at least about 135 contiguous amino acids of SEQ ID NO:1, at least about 140 contiguous amino acids of SEQ ID NO:1, at least about 145 contiguous amino acids of SEQ ID NO:1, at least about 150 contiguous amino acids of SEQ ID NO:1, at least about 55 contiguous amino acids of SEQ ID NO:1, at least about 160 contiguous amino acids of SEQ ID NO:1, at least about 165 contiguous amino acids of SEQ ID NO:1, at least about 170 contiguous amino acids of SEQ ID NO: 1, at least about 175 contiguous amino acids of SEQ ID NO:1, at least about 180 contiguous amino acids of SEQ ID NO:1, at least about 85 contiguous amino acids of SEQ ID NO:1, at least about 190 contiguous amino acids of SEQ ID NO:1, at least about 95 contiguous amino acids of SEQ ID NO:1, at least 200 contiguous amino acids of SEQ ID NO:1 or at least about 205 contiguous amino acids of SEQ ID NO:1.

In some embodiments, the nucleocapsid protein, fragment or variant thereof of the at least one fourth specific binding partner and the at least one eighth specific binding partner comprises a fusion protein comprising at least all or at least a portion of at least one SARS-CoV-2 isolated nucleocapsid protein or fragment or variant thereof. In another aspect, the fusion protein may comprise all or at least portion (e.g., at least 5 amino acids or more) of the NTD of a nucleocapsid protein from SARS-CoV-2 is operably linked, fused or grafted directly or indirectly (such as through one or more linking peptide sequences and/or HIS tags) to another protein or polypeptide. In still yet other aspects described herein, at least a portion (e.g., at least 5 amino acids or more) of a NTD of a spike protein from SARS-CoV-2 can be operably linked, fused or grafted directly or indirectly (such as through one or more linking peptide sequences and/or HIS tags) to all or at least a portion (at least 5 amino acids or more) of another protein or polypeptide. In another aspect, the fusion protein may comprise all or at least portion (e.g., at least 5 amino acids or more) of the CTD of a nucleocapsid protein from SARS-CoV-2 is operably linked, fused or grafted directly or indirectly (such as through one or more linking peptide sequences and/or HIS tags) to another protein or polypeptide.

In some embodiments, at least one of the fifth specific binding partner, sixth specific binding partner, seventh specific binding partner, or eighth specific binding partner can be immobilized on a solid support. In some embodiments, any or all of the at least one fifth specific binding partner, the at least one sixth specific binding partner, the at least one seventh specific binding partner, or the at least one eighth specific binding partner may be immobilized on the same solid support. In some embodiments, any or all of the at least one fifth specific binding partner, the at least one sixth specific binding partner, the at least one seventh specific binding partner, or the at least one eighth specific binding partner may be immobilized on different solid supports. In some embodiments, when any or all of the at least one fifth specific binding partner, the at least one sixth specific binding partner, the at least one seventh specific binding partner, or the at least one eighth specific binding partner are immobilized on the same solid support, the amount or ratio of the binding partners can be optimized for the amount of SARS-CoV-2 antigen or anti-SARS-CoV-2 antibody detected.

Upon contacting the sample with the at least one capture composition and the at least one detection composition, complexes may be formed comprising the first microparticle reagent and the first detection complex and/or the second microparticle reagent and the second detection reagent. The complex comprising the first microparticle reagent and the first detection complex is formed when the first microparticle reagent and the first detection complex each interact with a corresponding SARS-CoV-2 antigen, or fragment or variant thereof, in the sample. The complex comprising the second microparticle reagent and the second detection complex is formed when the second microparticle reagent and the second detection complex each interact with the same anti-SARS-CoV-2 antibody, or antibody fragment or variant thereof, in the sample.

In some embodiments, at least one first complex comprising the first specific binding partner-SARS-CoV-2-spike RBD antigen-fifth specific binding partner and a detectable label is produced. In some embodiments, at least one second complex comprising the second specific binding partner-SARS-CoV-2 nucleocapsid antigen-sixth specific binding partner and a detectable label. In some embodiments, at least one first complex comprising the first specific binding partner-SARS-CoV-2-spike RBD antigen-fifth specific binding partner and a detectable label and at least one second complex comprising the second specific binding partner-SARS-CoV-2 nucleocapsid antigen-sixth specific binding partner and a detectable label are produced.

In some embodiments, at least one third complex comprising the third specific binding partner-anti-SARS-CoV-2-spike RBD antibody-seventh specific binding partner and a detectable label is produced. In some embodiments, at least one fourth complex comprising the fourth specific binding-anti-SARS-CoV-2 nucleocapsid antibody-eighth specific binding partner and a detectable label. In some embodiments, at least one third complex comprising the third specific binding partner-anti-SARS-CoV-2-spike RBD antibody-seventh specific binding partner and a detectable label and at least one fourth complex comprising the fourth specific binding-anti-SARS-CoV-2 nucleocapsid antibody-eighth specific binding partner and a detectable label are produced.

In some embodiments, a sample may comprise one or more SARS-CoV-2 antigens or fragments or variants thereof in addition to one or more anti-SARS-CoV-2 antibodies or antibody fragments or variants thereof. As such, the methods described herein may result in the formation of one or more complexes to different SARS-CoV-2 antigens, or fragments or variants thereof and one or more complexes to different anti-SARS-CoV-2 antibodies, or antibody fragments or variants thereof. Thus, any combination of at least one first complex, at least one second complex, at least one third complex, and at least one fourth complex is possible utilizing the methods disclosed herein.

In some embodiments, a sample may comprise one or more SARS-CoV-2 antigens or fragments or variants thereof, and not comprise one or more anti-SARS-CoV-2 antibodies or antibody fragments or variants thereof. In such cases, at least one first complex and/or at least one second complex is formed depending on the nature of the SARS-CoV-2 antigens or fragments or variants thereof, as defined above for the at least one first complex and at least one second complex.

In some embodiments, a sample may comprise one or more of at least one type of anti-SARS-CoV-2 antibodies or antibody fragments or variants thereof, and not comprise one or more of at least one type of SARS-CoV-2 antigens or fragments or variants thereof. In such cases, at least one third complex and/or at least one fourth complex is formed depending on the nature of the at least one type of anti-SARS-CoV-2 antibodies, or antibody fragments or variants thereof, as defined above for the at least one third complex and at least one fourth complex.

The methods may further comprise assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant thereof in the sample. In some embodiments, the assessing the signal comprises detection of the presence or absence of the detectable labels on the at least one fifth specific binding partner, at least one sixth specific binding partner, at least one seventh specific binding partner, and at least one eighth specific binding partner.

The detectable labels on each of the at least one fifth specific binding partner, at least one sixth specific binding partner, at least one seventh specific binding partner, and at least one eighth specific binding partner may be the same or different. In some embodiments, the at least one fifth specific binding partner and the at least one sixth specific binding partner comprise the same detectable label, such that a positive result from either indicates the presence of at least one type SARS-CoV-2 antigen or fragment or variant thereof in the sample. In some embodiments, the at least one fifth specific binding partner and the at least one sixth specific binding partner comprise a different detectable label, such that a positive result from one or both indicates the presence of a SARS-CoV-2 spike protein antigen or fragment or variant and/or a SAS-CoV-2 nucleocapsid protein antigen or fragment or variant in the sample.

In some embodiments, the at least one seventh specific binding partner and the at least one eighth specific binding partner comprise the same detectable label, such that a positive result from either indicates the presence of at least one type of anti-SARS-CoV-2 antibody or antibody fragment or variant in the sample. In some embodiments, the at least one seventh specific binding partner and the at least one eighth specific binding partner comprise a different detectable label, such that a positive result from one or both indicates the presence of an anti-SARS-CoV-2 spike protein antibody or antibody fragment or variant thereof and/or an anti-SAS-CoV-2 nucleocapsid protein antibody or antibody fragment or variant in the sample.

In some embodiments, the signal from the first complex indicates the presence or amount of anti-SARS-CoV-2 spike RBD antigen or fragment or variant in the sample; the signal from the second complex indicates the presence or amount of anti-SARS-CoV-2 nucleocapsid antigen or fragment or variant in the sample; the signal from the third complex indicates the presence or amount of anti-SARS-CoV-2 spike RBD antibody or antibody fragment or variant thereof in the sample; and the signal from the fourth complex indicates the presence or amount of anti-SARS-CoV-2 nucleocapsid antibody or antibody fragment or variant in the sample.

The nature of methods described herein is not critical and can be conducted using any assay known in the art such as, for example, immunoassays, lateral flow assays, protein immunoprecipitation, immunoelectrophoresis, chemical analysis, SDS-PAGE and Western blot analysis, protein immunostaining, electrophoresis analysis, a protein assay, a competitive binding assay, or a functional protein assay. Also, the assay can be employed in a clinical chemistry format such as would be known by one of ordinary skill in the art and described herein. It is known in the art that the values (e.g., reference levels, cutoffs, thresholds, specificities, sensitivities, concentrations of calibrators and/or controls etc.) used in an assay that employs a specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

Other methods of detection include the use of, or can be adapted for use on, a nanopore device or nanowell device, e.g., for single molecule detection. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety. Other devices and methods appropriate for single molecule detection also can be employed.

In some embodiments, the biological sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

### Treatment and Monitoring of Subjects Identified as Having SARS-CoV-2

A subject identified according to the methods described above as having at least one type of anti-SARS-CoV-2 antibody or antibody fragment or variant thereof and at least one type of SARS-CoV-2 antigen or fragment or variant thereof and/or having a certain amount, concentration and/or level of at least one type of anti-SARS-CoV-2 antibody or antibody fragment or variant thereof and at least one type of SARS-CoV-2 antigen or fragment or variant thereof may be treated, monitored (e.g., by monitoring SARS-CoV-2 antigen or protein levels and/or anti-SARS-CoV-2 IgG and/or IgM antibody levels in the subject), treated and monitored and/or monitored and treated using routine techniques known in the art. Thus, in some embodiments, the method further comprises (a) monitoring the subject for SARS-CoV-2 IgA, SARS-CoV-2 IgG, and/or SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, and/or SARS-CoV-2 IgM antibodies and/or SARS-CoV-2 antigen and treating the subject for SARS-CoV-2; or (d) treating the subject for SARS-CoV-2 infection and monitoring the subject for SARS-CoV-2 IgA, SARS-CoV-2 IgG, and/or SARS-CoV-2 IgM antibodies and/or SARS-CoV-2 antigen. In other embodiments, the methods described herein further include treating the subject (e.g., such as a human) identified as having at least one type of anti-SARS-CoV-2 antibody and at least one type of SARS-CoV-2 antigen and/or having a certain amount, concentration and/or level of at least one type of anti-SARS-CoV-2 antibody and at least one type of SARS-CoV-2 antigen in one or more biological samples obtained from the subject.

The treatment can take a variety of forms depending on whether or not the subject is asymptomatic or experiencing mild, moderate or severe infection with SARS-CoV-2. For example, subjects experiencing mild infection with SARS-CoV-2, will experience a fever, cough (with or without sputum production), anorexia, malaise, muscle pain, sore throat, dyspnea, nasal congestion, headache, diarrhea, nausea, and vomiting or any combination thereof. Subjects experiencing a moderate infection will experience a fever greater than 100.4° F. that lasts for several days, chills, shortness of breath, lethargy, or any combination thereof. Such subjects may be suffering from pneumonia. Subjects experiencing severe infection will experience trouble breathing, persistent pain or pressure in the chest, confusion, inability to rouse, bluish lips or face, or any combination thereof. Such subjects may be suffering from severe pneumonia.

If the subject is asymptomatic or has mild symptoms, the subject may be treated with rest and sleep, by keeping warm, ingesting fluids (e.g., remaining hydrated) minimizing social interaction with other subjects (e.g., remain isolated or quarantined, such as, for example, at home), or any combination thereof. Additionally, the subject can be monitored to see if symptoms arise and/or worsen.

Subjects with moderate or severe symptoms of infection with SARS-CoV-2, may be treated with one or more drugs, vaccines, convalescent plasma therapy (e.g., receiving plasma from blood taken from a subject that has survived an infection with SARS-CoV-2, or respiratory support or assistance (e.g., receiving supplemental oxygen through a nasal cannula, nasal prongs, face mask, or non-invasive or invasive (e.g. intubation) ventilation) or combinations thereof. Examples of one or more drugs that can be used to treat a subject include, but are not limited to, remdesivir, hydroxychloroquine, chloroquine or combinations thereof. Subjects receiving any of the aforementioned treatment also can further be monitored using routine techniques known in the art.

In other embodiments, a subject may be monitored prior to being treated for SARS-CoV-2. Such monitoring involves detecting, analyzing and/or interpreting changes in the subject's SARS-CoV-2 antigen or protein levels and/or anti-SARS-CoV-2 IgG and/or IgM antibody levels over the course of time. For example, depending on a subject's SARS-CoV-2 antigen or protein levels and/or SARS-CoV-2 IgM antibody levels, a subject may be monitored prior to receiving any treatment to gauge whether the subject's immune system is able to fight the virus on its own without any treatment intervention. During the course of the monitoring, if the subject's SARS-CoV-2 antigen or protein levels and/or SARS-CoV-2 IgM antibody levels increase, treatment can be commenced. Likewise, during treatment, a subject's SARS-CoV-2 IgM and/or IgG antibody levels can be monitored. If during treatment the subject's SARS-CoV-2 IgM antibody levels remain high and SARS-CoV-2 IgG antibody levels remain low, the subject can be continued to be treated for SARS-CoV-2 and continued to monitored until such time that the subject's SARS-CoV-2 IgM antibody levels have lowered and SARS-CoV-2 IgG antibody levels increased.

In other embodiments, monitoring a subject for SARS-CoV-2 antigen may comprise the use of laboratory tests such as, for example, polymerase chain reaction (PCR) or other nucleic acid amplification-based assays (also referred to as “molecular” tests), serology or antibody assays, and antigen assays. Molecular tests detect the genetic material or nucleic acid present inside a virus particle. Most molecular tests employ PCR-based methods (e.g., RT-PCR), which are also referred to as nucleic acid amplification tests (NAAT). The FDA has authorized molecular tests for use in clinical laboratory settings and authorized some for use in a point-of-care (POC) setting, including, for example, ID NOW™ COVID-19, REALTIME™ SARS-CoV-2 EUA, and ALINITY™ m SARS-CoV-2 assay (all marketed by Abbott Laboratories, Abbott Park, IL). Thus, monitoring a subject or SARS-CoV-2 antigen may comprise detecting SARS-CoV-2 viral RNA using PCR. In some embodiments, monitoring a subject or SARS-CoV-2 antigen may comprise determining the presence of SARS-CoV-2 viral RNA using PCR. In some embodiments, monitoring a subject or SARS-CoV-2 antigen may comprise determining the presence of a SARS-CoV-2 viral antigen.

In some embodiments, the subject may have been previously confirmed as having COVID-19 or an infection by SARS-CoV-2 such that the assay described herein is used to monitor SARS-CoV-2 antigen or protein levels and/or anti-SARS-CoV-2 IgG and/or IgM antibody levels in the subject.

The above-described methods may be performed in any suitable time period. Ideally, the method for detecting a presence or determining an amount of at least one SARS-CoV-2 antigen in a subject is performed in from about 5 to about 30 minutes (e.g., about 5, 6, 7, 8, 9, 10, 11, 12, 13, 14, 15, 16, 17, 18, 19, 20, 21, 22, 23, 24, 25, 26, 27, 28, 29 or 30 minutes, or a range defined by any two of the foregoing values). For example, the method may be performed in about 5-10 minutes, about 5-20 minutes, about 7-12 minutes, about 10-15 minutes, about 15-20 minutes, or about 15-30 minutes.

### Preparation/Productions Methods for Recombinant Antigens for Use as Specific Binding Partners

Once sequenced, polypeptides, such as a nucleocapsid protein, fragment or variant thereof of SARS-CoV-2; a spike protein, fragment or variant thereof from SARS-CoV-2; any protein, fragment or variant thereof that binds to an anti-nucleocapsid antibody or antibody fragment or variant thereof; and/or any protein, fragment or variant thereof that binds to any anti-spike antibody or antibody fragment or variant thereof, can be synthesized using methods known in the art, such as, for example, exclusive solid phase synthesis, partial solid phase synthesis, fragment condensation, and classical solution synthesis. See, e.g., Merrifield, J. Am. Chem. Soc. 85: 2149 (1963). On a solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the tradename BIO-BEADS SX-1 by Bio Rad Laboratories (Richmond, Calif.), and the preparation of the hydroxymethyl resin is described by Bodonszky et al., Chem. Ind. (London) 38: 1597 (1966). The benzhydrylamine (BHA) resin has been described by Pietta and Marshall, Chem. Comm 650 (1970) and is commercially available from Beckman Instruments, Inc. (Palo Alto, Calif.) in the hydrochloride form. Automated peptide synthesizers are commercially available, as are services that make peptides to order.

Thus, the polypeptides can be prepared by coupling an alpha-amino protected amino acid to the chloromethylated resin with the aid of, for example, cesium bicarbonate catalyst, according to the method described by Gisin, Hely. Chim. Acta. 56: 1467 (1973). After the initial coupling, the alpha-amino protecting group is removed by a choice of reagents including trifluoroacetic acid (TFA) or hydrochloric acid (HCl) solutions in organic solvents at room temperature.

Suitable alpha-amino protecting groups include those known to be useful in the art of stepwise synthesis of peptides. Examples of alpha-amino protecting groups are: acyl type protecting groups (e.g., formyl, trifluoroacetyl, and acetyl), aromatic urethane type protecting groups (e.g., benzyloxycarbonyl (Cbz) and substituted Cbz), aliphatic urethane protecting groups (e.g., t-butyloxycarbonyl (Boc), isopropyloxycarbonyl, and cyclohexyloxycarbonyl), and alkyl type protecting groups (e.g., benzyl and triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride and dimethyl formamide (DMF) mixtures.

After the desired amino acid sequence has been completed, the desired peptide is decoupled from the resin support by treatment with a reagent, such as TFA or hydrogen fluoride (HF), which not only cleaves the peptide from the resin, but also cleaves all remaining side chain protecting groups. When the chloromethylated resin is used, HF treatment results in the formation of the free peptide acids. When the benzhydrylamine resin is used, HF treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These and other solid phase peptide synthesis procedures are well-known in the art. Such procedures are also described by Stewart and Young in Solid Phase Peptide Syntheses (2nd Ed., Pierce Chemical Company, 1984).

All or a portion of the nucleocapsid protein, a fragment or variant thereof from SARS-CoV-2; the spike protein, a fragment or variant thereof from SARS-CoV-2; any protein, fragment, variant thereof that binds to an anti-nucleocapsid antibody or antibody fragment or variant thereof from SARS-CoV-2; and/or any protein, fragment, or variant thereof that binds to any anti-spike antibody or antibody fragment or variant thereof from SARS-CoV-2 can be recombinantly produced using methods known in the art. For example, an isolated or purified nucleic acid molecule comprising a nucleotide sequence encoding the polypeptide can be expressed in a host cell, and the polypeptide can be isolated. The isolated or purified nucleic acid molecule can comprise a nucleotide sequence encoding the nucleocapsid protein, a fragment or variant thereof from SARS-CoV-2; spike protein, a fragment or variant thereof from SARS-CoV-2; any protein, fragment, or variant thereof that binds to an anti-nucleocapsid antibody or antibody fragment or variant thereof from SARS-CoV-2; and/or any protein, fragment, or variant thereof that binds to any anti-spike antibody or antibody fragment or variant thereof from SARS-CoV-2.

In some embodiments, the isolated or purified nucleic acid molecule comprises a nucleotide sequence encoding the RBD of a spike protein from SARS-CoV-2, or a fragment or variant thereof that specifically binds to at least one anti-SARS-CoV-2 spike RBD antibody or antibody fragment or variant thereof. In some embodiments, the isolated or purified nucleic acid molecule comprises a nucleotide sequence encoding a RBD from a spike protein having the amino acid sequence of SEQ ID NOS: 5 or 6. In some embodiments, the isolated or purified nucleic acid molecule comprises a nucleotide sequence encoding the nucleocapsid protein from SARS-CoV-2, or a fragment or variant thereof (e.g. the C-terminal domain) that specifically binds to at least one anti-SARS-CoV-2 nucleocapsid antibody or antibody fragment or variant thereof. In some embodiments, the isolated or purified nucleic acid molecule can comprise a nucleic acid sequence encoding a nucleocapsid protein having the amino acid sequence of SEQ ID NO:1 or a fragment or variant thereof.

The isolated nucleic acid can be synthesized with an oligonucleotide synthesizer, for example. One of ordinary skill in the art will readily appreciate that, due to the degeneracy of the genetic code, more than one nucleotide sequence can encode a given amino acid sequence. Codons, which are favored by a given host cell, preferably are selected for recombinant production. A nucleotide sequence encoding the amino acid sequence of a specified sequence can be combined with other nucleotide sequences using polymerase chain reaction (PCR), ligation, or ligation chain reaction (LCR) to encode a mutated truncated nucleocapsid and/or spike polypeptide. The individual oligonucleotides typically contain 5′ or 3′ overhangs for complementary assembly. Once assembled, the nucleotide sequence encoding the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 can be inserted into a vector, operably linked to control sequences as necessary for expression in a given host cell, and introduced (such as by transformation or transfection) into a host cell. The nucleotide sequence can be further manipulated (for example, linked to one or more nucleotide sequences encoding additional immunoglobulin domains, such as additional constant regions) and/or expressed in a host cell.

Although not all vectors and expression control sequences may function equally well to express a polynucleotide sequence of interest and not all hosts function equally well with the same expression system, it is believed that those skilled in the art will be able to make a selection among these vectors, expression control sequences, optimized codons, and hosts for use in the present disclosure without any undue experimentation. For example, in selecting a vector, the host must be considered because the vector must be able to replicate in it or be able to integrate into the chromosome. The vector's copy number, the ability to control that copy number, and the expression of any other proteins encoded by the vector, such as antibiotic markers, should also be considered. In selecting an expression control sequence, a variety of factors also can be considered. These include, but are not limited to, the relative strength of the sequence, its controllability, and its compatibility with the nucleotide sequence encoding the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2, particularly with regard to potential secondary structures. Hosts should be selected by consideration of their compatibility with the chosen vector, their codon usage, their secretion characteristics, their ability to fold the polypeptide correctly, their fermentation or culture requirements, their ability (or lack thereof) to glycosylate the protein, and the ease of purification of the products encoded by the nucleotide sequence, etc.

The recombinant vector can be an autonomously replicating vector, namely, a vector existing as an extrachromosomal entity, the replication of which is independent of chromosomal replication (such as a plasmid). Alternatively, the vector can be one which, when introduced into a host cell, is integrated into the host cell genome and replicated together with the chromosome(s) into which it has been integrated.

The vector is preferably an expression vector in which the polynucleotide sequence encoding the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2, is operably linked to additional segments required for transcription of the polynucleotide sequence. The vector is typically derived from plasmid or viral DNA. A number of suitable expression vectors for expression in the host cells mentioned herein are commercially available or described in the literature. Useful expression vectors for eukaryotic hosts, include, but are not limited to, vectors comprising expression control sequences from SV40, bovine papilloma virus, adenovirus and cytomegalovirus. Specific vectors include pcDNA3.1 (+)\Hyg (Invitrogen Corp., Carlsbad, Calif.) and pCI-neo (Stratagene, La Jolla, Calif.). Examples of expression vectors for use in yeast cells include, but are not limited to, the 2p plasmid and derivatives thereof, the POT1 vector (see, e.g., U.S. Pat. No. 4,931,373), the pJSO37 vector (described in Okkels, Ann New York Acad. Sci. 782: 202-207 (1996)) and pPICZ A, B or C (Invitrogen). Examples of expression vectors for use in insect cells include, but are not limited to, pVL941, pBG311 (Cate et al., Cell 45: 685-698 (1986)), and pBluebac 4.5 and pMelbac (both of which are available from Invitrogen).

Other vectors that can be used allow the nucleotide sequence encoding the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2, to be amplified in copy number. Such amplifiable vectors are well-known in the art. These vectors include, but are not limited to, those vectors that can be amplified by dihydrofolate reductase (DHFR) amplification (see, for example, U.S. Pat. No. 4,470,461 and Kaufinan et al., Mol. Cell. Biol. 2: 1304-1319 (1982)) and glutamine synthetase (GS) amplification (see, for example, U.S. Pat. No. 5,122,464 and EP Patent Application Publication No. 0 338 841).

The recombinant vector can further comprise a nucleotide sequence enabling the vector to replicate in the host cell in question. An example of such a sequence for use in a mammalian host cell is the SV40 origin of replication. Suitable sequences enabling the vector to replicate in a yeast cell are the yeast plasmid 2p replication genes REP 1-3 and origin of replication.

The vector can also comprise a selectable marker, namely, a gene or polynucleotide, the product of which complements a defect in the host cell, such as the gene coding for DHFR or the Schizosaccharomyces pombe TPI gene (see, e.g., Russell, Gene 40: 125-130 (1985)), or one which confers resistance to a drug, such as ampicillin, kanamycin, tetracycline, chloramphenicol, neomycin, hygromycin or methotrexate. For filamentous fungi, selectable markers include, but are not limited to, amdS, pyrG, arcB, niaD and sC.

Also present in the vector are “control sequences,” which are any components that are necessary or advantageous for the expression of the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2. Each control sequence can be native or foreign to the nucleotide sequence encoding the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2. Such control sequences include, but are not limited to, a leader, a polyadenylation sequence, a propeptide sequence, a promoter, an enhancer or an upstream activating sequence, a signal peptide sequence, and a transcription terminator. At a minimum, the control sequences include at least one promoter operably linked to the polynucleotide sequence encoding the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2.

By “operably linked” is meant the covalent joining of two or more nucleotide sequences, by means of enzymatic ligation or otherwise, in a configuration relative to one another such that the normal function of the sequences can be performed. For example, a nucleotide sequence encoding a presequence or secretory leader is operably linked to a nucleotide sequence for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in the same reading frame. Linking is accomplished by ligation at convenient restriction sites. If such sites do not exist, then synthetic oligonucleotide adaptors or linkers can be used, in conjunction with standard recombinant DNA methods.

A wide variety of expression control sequences can be used in the context of the present disclosure. Such useful expression control sequences include the expression control sequences associated with structural genes of the foregoing expression vectors as well as any sequence known to control the expression of genes of prokaryotic or eukaryotic cells or their viruses, and various combinations thereof. Examples of suitable control sequences for directing transcription in mammalian cells include the early and late promoters of SV40 and adenovirus, for example, the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak, J. Mol. Biol. 196: 947-50 (1987)).

In order to improve expression in mammalian cells a synthetic intron can be inserted in the 5′ untranslated region of a polynucleotide sequence encoding the antibody or an antibody fragment thereof. An example of a synthetic intron is the synthetic intron from the plasmid pCI-Neo (available from Promega Corporation, Madison, Wis.).

Examples of suitable control sequences for directing transcription in insect cells include, but are not limited to, the polyhedrin promoter, the P10 promoter, the baculovirus immediate early gene 1 promoter, the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4-c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulas glucoamylase, A. nidulans acetamidase, Rhizomucor miehei aspartic proteinase or lipase, the TPI1 terminator, and the ADH3 terminator.

The polynucleotide sequence encoding the truncated DBP, mutated truncated DBP, or fusion protein of either of the foregoing may or may not also include a polynucleotide sequence that encodes a signal peptide. The signal peptide is present when the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 is to be secreted from the cells in which it is expressed. Such signal peptide, if present, should be one recognized by the cell chosen for expression of the polypeptide. The signal peptide can be homologous or heterologous to the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 can be homologous or heterologous to the host cell, i.e., a signal peptide normally expressed from the host cell or one which is not normally expressed from the host cell. Accordingly, the signal peptide can be prokaryotic, for example, derived from a bacterium, or eukaryotic, for example, derived from a mammalian, insect, filamentous fungal, or yeast cell.

The presence or absence of a signal peptide will, for example, depend on the expression host cell used for the production of the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2. For use in filamentous fungi, the signal peptide can conveniently be derived from a gene encoding an Aspergillus sp. amylase or glucoamylase, a gene encoding a Rhizomucor miehei lipase or protease or a Humicola lanuginosa lipase. For use in insect cells, the signal peptide can be derived from an insect gene (see, e.g., PCT International Application WO 90/05783), such as the lepidopteran Manduca sexta adipokinetic hormone precursor (see, e.g., U.S. Pat. No. 5,023,328), the honeybee melittin (Invitrogen), ecdysteroid UDP glucosyltransferase (egt) (Murphy et al., Protein Expression and Purification 4: 349-357 (1993), or human pancreatic lipase (hpl) (Methods in Enzymology 284: 262-272 (1997)).

Specific examples of signal peptides for use in mammalian cells include murine Ig kappa light chain signal peptide (Coloma, J. Imm Methods 152: 89-104 (1992)). Suitable signal peptides for use in yeast cells include the α-factor signal peptide from S. cerevisiae (see, e.g., U.S. Pat. No. 4,870,008), the signal peptide of mouse salivary amylase (see, e.g., Hagenbuchle et al., Nature 289: 643-646 (1981)), a modified carboxypeptidase signal peptide (see, e.g., Valls et al., Cell 48: 887-897 (1987)), the yeast BAR1 signal peptide (see, e.g., PCT International Application WO 87/02670), and the yeast aspartic protease 3 (YAPS) signal peptide (see, e.g., Egel-Mitani et al., Yeast 6: 127-137 (1990)).

In view of the above, the above-described isolated or purified nucleic acid molecule, which can be a vector, can be introduced into a host cell as described herein below. Accordingly, a host cell comprising the isolated or purified nucleic acid molecule is provided.

Any suitable host can be used to produce the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2, including bacteria, fungi (including yeasts), plant, insect, mammal or other appropriate animal cells or cell lines, as well as transgenic animals or plants. A preferred host cell is a Chinese hamster ovary (CHO) cell. Examples of bacterial host cells include, but are not limited to, gram-positive bacteria, such as strains of Bacillus, for example, B. brevis or B. subtilis, Pseudomonas or Streptomyces, or gram-negative bacteria, such as strains of E. coli. The introduction of a vector into a bacterial host cell can, for instance, be affected by protoplast transformation (see, for example, Chang et al., Molec. Gen. Genet. 168: 111-115 (1979)), using competent cells (see, for example, Young et al., J. of Bacteriology 81: 823-829 (1961), or Dubnau et al., J. of Molec. Biol. 56: 209-221 (1971)), electroporation (see, for example, Shigekawa et al., Biotechniques 6: 742-751 (1988)), or conjugation (see, for example, Koehler et al., J. of Bacteriology 169: 5771-5278 (1987)).

Examples of suitable filamentous fungal host cells include, but are not limited to, strains of Aspergillus, for example, A. oryzae, A. niger, or A. nidulans, Fusarium or Trichoderma. Fungal cells can be transformed by a process involving protoplast formation, transformation of the protoplasts, and regeneration of the cell wall using techniques known to those ordinarily skilled in the art. Suitable procedures for transformation of Aspergillus host cells are described in EP Patent Application No. 0 238 023 and U.S. Pat. No. 5,679,543. Suitable methods for transforming Fusarium species are described by Malardier et al., Gene 78: 147-156 (1989), and PCT International Application WO 96/00787. Yeast can be transformed using the procedures described by Becker and Guarente, In Abelson, J. N. and Simon, M. I., editors, Guide to Yeast Genetics and Molecular Biology, Methods in Enzymology 194: 182-187, Academic Press, Inc., New York; Ito et al, J. of Bacteriology 153: 163 (1983); and Hinnen et al., PNAS USA 75: 1920 (1978).

Examples of suitable yeast host cells include strains of Saccharomyces, for example, S. cerevisiae, Schizosaccharomyces, Klyveromyces, Pichia, such as P. pastoris or P. methanolica, Hansenula, such as H. polymorpha or yarrowia. Methods for transforming yeast cells with heterologous polynucleotides and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters 99: 193-198 (1992), Manivasakam et al., Nucleic Acids Research 21: 4414-4415 (1993), and Ganeva et al., FEMS Microbiology Letters 121: 159-164 (1994).

Examples of suitable insect host cells include, but are not limited to, a Lepidoptora cell line, such as Spodoptera frugiperda (Sf9 or Sf21) or Trichoplusia ni cells (High Five) (see, e.g., U.S. Pat. No. 5,077,214). Transformation of insect cells and production of heterologous polypeptides are well-known to those skilled in the art.

Examples of suitable mammalian host cells include Chinese hamster ovary (CHO) cell lines, simian (e.g., Green Monkey) cell lines (COS), mouse cells (for example, NS/O), baby hamster kidney (BHK) cell lines, human cells (such as human embryonic kidney (HEK) cells (e.g., HEK 293 cells (A.T.C.C. Accession No. CRL-1573)), myeloma cells that do not otherwise produce immunoglobulin protein, and plant cells in tissue culture. Preferably, the mammalian host cells are CHO cell lines and/or HEK (e.g., HEK 293) cell lines. Another preferred host cell is the B3.2 cell line (e.g., AbbVie, AbbVie Bioresearch Center, Worcester, Mass.), or another dihydrofolate reductase deficient (DHFR−) CHO cell line (e.g., available from Invitrogen).

Methods for introducing exogenous polynucleotides into mammalian host cells include calcium phosphate-mediated transfection, electroporation, DEAE-dextran mediated transfection, liposome-mediated transfection, viral vectors, and the transfection method described by Life Technologies Ltd, Paisley, UK using Lipofectamine™ 2000. These methods are well-known in the art and are described, for example, by Ausbel et al. (eds.), Current Protocols in Molecular Biology, John Wiley & Sons, New York, USA (1996). The cultivation of mammalian cells is conducted according to established methods, e.g., as disclosed in Jenkins, Ed., Animal Cell Biotechnology, Methods and Protocols, Human Press Inc. Totowa, N.J., USA (1999), and Harrison and Rae, General Techniques of Cell Culture, Cambridge University Press (1997).

In the production methods, cells are cultivated in a nutrient medium suitable for production of the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 using methods known in the art. For example, cells are cultivated by shake flask cultivation, small-scale or large-scale fermentation (including continuous, batch, fed-batch, or solid state fermentations) in laboratory or industrial fermenters performed in a suitable medium and under conditions the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 to be expressed and/or isolated. The cultivation takes place in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection). If the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 is secreted into the nutrient medium, it can be recovered directly from the medium. If the truncated DBP (or fusion protein thereof) or mutated truncated DBP (or fusion protein thereof) is not secreted, it can be recovered from cell lysates.

The resulting nucleocapsid protein, a fragment or variant thereof from SARS-CoV-2; spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 can be recovered by methods known in the art. For example, the nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 can be recovered from the nutrient medium by conventional procedures including, but not limited to, centrifugation, filtration, extraction, spray drying, evaporation, or precipitation.

The nucleocapsid protein, a fragment or variant thereof from SARS-CoV-2; the spike protein, a fragment or variant thereof from SARS-CoV-2; any protein, fragment or variant thereof that binds to an anti-nucleocapsid antibody or antibody fragment or variant thereof from SARS-CoV-2; and/or any protein, fragment or variant thereof that binds to any anti-spike antibody or antibody fragment or variant thereof from a SARS-CoV-2 can be purified by a variety of procedures known in the art including, but not limited to, chromatography (such as, but not limited to, ion exchange, affinity, hydrophobic, chromatofocusing, and size exclusion), electrophoretic procedures (such as, but not limited to, preparative isoelectric focusing), differential solubility (such as, but not limited to, ammonium sulfate precipitation), SDS-PAGE, or extraction (see, for example, Janson and Ryden, editors, Protein Purification, VCH Publishers, New York (1989)). In some embodiments, the purification can be done in CHO and/or HEK cells using routine techniques known in the art. In other embodiments, when purifying the RBD of spike, any monomeric RBD can be separated from any dimeric RBD using routine techniques known in the art such as, for example, affinity chromatography, gel filtration chromatography, ion-exchange chromatography, high-pressure liquid chromatography, etc. In some embodiments, the purification is done using immobilized metal affinity chromatography (IMAC), such as, for example, as described in “Block et al., “Chapter 27 Immobilized-Metal Affinity Chromatography (IPAC): A Review”, Methods in Enzymology, 463:439-473 (2009) and Spriestersbach et al., “Chapter One—Purification of His-Tagged Proteins”, Methods in Enzymology, 559:1-15 (2015), the contents of which are herein incorporated by reference.

### Preparation/Production Methods for Antibodies for Use as Specific Binding Partners

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including DHFR-CHO cells, described in Urlaub and Chasin, Proc. Natl. Acad. Sci. USA, 77: 4216-4220 (1980)), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. In some embodiments, the purification of the antibodies can be done in CHO and/or HEK cells using routine techniques known in the art.

Host cells also can be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are a human β-coronavirus antibody (i.e., binds to one or more epitopes on a β-coronavirus, such as SARS-CoV or SARS-CoV-2) and the other heavy and light chain are specific for an antigen other than a human β-coronavirus (e.g., such as SARS-CoV or SARS-CoV-2) by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into DHFR-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with β-coronavirus (e.g., such as SARS-CoV or SARS-CoV-2) or a fragment (e.g., such as from the nucleocapsid and/or spike proteins) and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) Biolnvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass); Gray et al. (1995) J. Imm. Meth. 182:155-163; Kenny et al. (1995) Bio/Technol. 13:787-790); and B-cell selection (Steenbakkers et al. (1994) Molec. Biol. Reports 19:125-134 (1994)).

An affinity matured antibody may be produced by any one of a number of procedures that are known in the art. For example, Marks et al., BioTechnology, 10: 779-783 (1992) describes affinity maturation by VH and VL domain shuffling. Random mutagenesis of CDR and/or framework residues is described in Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); Hawkins et al, J. Mol. Biol., 226: 889-896 (1992). Selective mutation at selective mutagenesis positions and at contact or hypermutation positions with an activity enhancing amino acid residue is described in U.S. Pat. No. 6,914,128 B1.

Antibody fragments or variants thereof also can be prepared by delivering a polynucleotide encoding an antibody to a suitable host, so as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody fragments or variants thereof also can be prepared by delivering a polynucleotide to provide transgenic plants and cultured plant cells (e.g., tobacco, maize, and duckweed) that produce such antibodies, specified portions or variants in the plant parts or in cells cultured therefrom. For example, Cramer et al. (1999) Curr. Top. Microbiol. Immunol. 240:95-118, and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody fragments or variants thereof have also been produced in large amounts from transgenic plant seeds including antibody fragments, such as single chain antibodies (scFv's), using, for example, tobacco seeds and potato tubers. See, e.g., Conrad et al. (1998) Plant Mol. Biol. 38:101-109 and references cited therein. Thus, antibodies also can be produced using transgenic plants according to known methods.

Antibody derivatives can be produced, for example, by adding exogenous sequences to modify immunogenicity or to reduce, enhance, or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein such fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al., which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxyapatite chromatography, and lectin chromatography. High performance liquid chromatography (“HPLC”) also can be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

Antibody production via the use of hybridoma technology, the selected lymphocyte antibody method (SLAM), transgenic animals, and recombinant antibody libraries is described in more detail below.

Monoclonal Antibodies Using Hybridoma Technology. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody wherein, preferably, the hybridoma is generated by fusing splenocytes isolated from an animal, e.g., a rat or a mouse, immunized with a β-coronavirus, such as SARS-CoV or SARS-CoV-2 (e.g., such as a human, mouse, rat, rabbit SARS-CoV or SARS-CoV-2), or a fragment or variant with myeloma cells and then screening the hybridomas resulting from the fusion for hybridoma clones that secrete an antibody able to bind a polypeptide. Briefly, rats can be immunized with a β-coronavirus (such as SARS-CoV or SARS-CoV-2) antigen. In a preferred embodiment, the β-coronavirus (such as SARS-CoV or SARS-CoV-2) antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with at least one type of β-coronavirus (such as SARS-CoV or SARS-CoV-2) antigen, antibodies and/or antibody-producing cells may be obtained from the animal. An anti-3-coronavirus (such as SARS-CoV or SARS-CoV-2) antibody-containing serum is obtained from the animal by bleeding or sacrificing the animal. The serum may be used as it is obtained from the animal, an immunoglobulin fraction may be obtained from the serum, or the anti-3-coronavirus (such as SARS-CoV or SARS-CoV-2) antibodies may be purified from the serum. Serum or immunoglobulins obtained in this manner are polyclonal, thus having a heterogeneous array of properties.

Once an immune response is detected, e.g., antibodies specific for the at least one type of β-coronavirus (such as SARS-CoV or SARS-CoV-2) antigen are detected in the rat serum, the rat spleen is harvested, and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding to at least one type of β-coronavirus (such as, for example, SARS-CoV or SARS-CoV-2). Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using at least one type of β-coronavirus (such as SARS-CoV or SARS-CoV-2), or a portion thereof, or a cell expressing at least one type of β-coronavirus (such as SARS-CoV or SARS-CoV-2) or portion thereof. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

β-coronavirus (such as SARS-CoV or SARS-CoV-2) antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning, and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another embodiment, the hybridomas may be human hybridomas, in which a human non-secretory myeloma is fused with a human cell expressing at least one type of anti-β-coronavirus (such as anti-SARS-CoV or anti-SARS-CoV-2) antibody.

Antibody fragments that recognize specific epitopes may be generated by known techniques. For example, Fab and F(ab′)₂ fragments may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

Monoclonal Antibodies Using SLAM. In another aspect, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93: 7843-7848 (1996). In this method, single cells secreting antibodies of interest, e.g., lymphocytes derived from an immunized animal, are screened using an antigen-specific hemolytic plaque assay, wherein the at least one type β-coronavirus (such as SARS-CoV or SARS-CoV-2) antigen is coupled to sheep red blood cells using a linker, such as biotin, and used to identify single cells that secrete antibodies with specificity for at least one type of β-coronavirus (such as SARS-CoV or SARS-CoV-2). Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to at least one type of β-coronavirus (such as SARS-CoV or SARS-CoV-2). The amplified immunoglobulin sequences further can be manipulated in vitro, such as by an in vitro affinity maturation method. See, for example, PCT Publication No. WO 97/29131 and PCT Publication No. WO 00/56772.

Monoclonal Antibodies Using Transgenic Animals. In another embodiment, antibodies may be produced by immunizing a non-human animal comprising some, or all, of the human immunoglobulin locus with at least one type of β-coronavirus (such as SARS-CoV or SARS-CoV-2) antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7: 13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15: 146-156 (1997), Green and Jakobovits, J. Exp. Med., 188: 483-495 (1998), the disclosures of which are hereby incorporated by reference.

Monoclonal Antibodies Using Recombinant Antibody Libraries. In vitro methods also can be used to make the antibodies, wherein an antibody library is screened to identify an antibody having the desired β-coronavirus (such as SARS-CoV or SARS-CoV-2)-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9: 1369-1372 (1991); Hay et al., Hum. Antibod. Hybridomas, 3: 81-85 (1992); Huse et al., Science, 246: 1275-1281 (1989); McCafferty et al., Nature, 348: 552-554 (1990); Griffiths et al., EMBO J., 12: 725-734 (1993); Hawkins et al., J. Mol. Biol., 226: 889-896 (1992); Clackson et al., Nature, 352: 624-628 (1991); Gram et al., Proc. Natl. Acad. Sci. USA, 89: 3576-3580 (1992); Garrard et al., Bio/Technology, 9: 1373-1377 (1991); Hoogenboom et al., Nucl. Acids Res., 19: 4133-4137 (1991); Barbas et al., Proc. Natl. Acad. Sci. USA, 88: 7978-7982 (1991); U.S. Patent Application Publication No. 2003/0186374; and PCT Publication No. WO 97/29131, the contents of each of which are incorporated herein by reference.

The recombinant antibody library may be from a subject immunized with at least one type of β-coronavirus (such as SARS-CoV or SARS-CoV-2) antigen. Alternatively, the recombinant antibody library may be from a naive subject, i.e., one who has not been immunized with at least one type of β-coronavirus (such as SARS-CoV or SARS-CoV-2) antigen, such as a human antibody library from a human subject who has not been immunized with at least one type of human β-coronavirus (such as SARS-CoV or SARS-CoV-2) antigen. Antibodies are selected by screening the recombinant antibody library with the peptide comprising human β-coronavirus (such as SARS-CoV or SARS-CoV-2) or fragment or variant thereof to thereby select those antibodies that recognize the at least one type of β-coronavirus (e.g., SARS-CoV or SARS-CoV-2) of interest. Methods for conducting such screening and selection are well known in the art, such as described in the references in the preceding paragraph. To select antibodies having particular binding affinities, the art-known method of surface plasmon resonance can be used to select antibodies having the desired K_{off} rate constant. To select antibodies having a particular neutralizing activity for at least one type of β-coronavirus, such as SARS-CoV or SARS-CoV-2, such as those with a particular IC₅₀, standard methods known in the art for assessing the inhibition of at least one type of β-coronavirus (such as SARS-CoV or SARS-CoV-2) activity may be used.

For example, antibodies also can be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used in the context of the present disclosure include those described in Brinkmann et al., J. Immunol. Methods, 182: 41-50 (1995); Ames et al., J. Immunol. Methods, 184:177-186 (1995); Kettleborough et al., Eur. J. Immunol., 24: 952-958 (1994); Persic et al., Gene, 187: 9-18 (1997); Burton et al., Advances in Immunology, 57: 191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments also can be employed using methods known in the art such as those disclosed in PCT publication No. WO 92/22324; Mullinax et al., BioTechniques, 12(6): 864-869 (1992); Sawai et al., Am. J. Reprod. Immunol., 34: 26-34 (1995); and Better et al., Science, 240: 1041-1043 (1988). Examples of techniques which can be used to produce single-chain Fvs and antibodies include those described in U.S. Pat. Nos. 4,946,778 and 5,258,498; Huston et al., Methods in Enzymology, 203: 46-88 (1991); Shu et al., Proc. Natl. Acad. Sci. USA, 90: 7995-7999 (1993); and Skerra et al., Science, 240: 1038-1041 (1988).

Alternative to screening of recombinant antibody libraries by phage display, other methodologies known in the art for screening large combinatorial libraries can be applied to the identification of antibodies. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94: 12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies also can be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

Production of Recombinant Antibodies. Recombinant antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing recombinant antibodies include Chinese Hamster Ovary (CHO cells) (including dhfr-CHO cells, described in Urlaub and Chasin, Proc. Natl. Acad. Sci. USA, 77: 4216-4220 (1980), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. In some embodiments, the antibodies can be purified in CHO and/or HEK cells using routine techniques known in the art.

Host cells also can be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds to an IgG antibody, IgM antibody and/or IgG or IgM antibody) and the other heavy and light chain are specific for an antigen other than an IgG antibody, IgM antibody and/or an IgG and IgM antibody by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody by culturing a host cell in a suitable culture medium until a recombinant antibody is synthesized. The method can further comprise isolating the recombinant antibody from the culture medium.

Humanized Antibody The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for a β-coronavirus (such as SARS-CoV and SARS-CoV-2) and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for at least one type of β-coronavirus (such as SARS-CoV and SARS-CoV-2), is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g., mice that are capable, upon immunization, of producing a full repertoire of human antibodies in the absence of endogenous immunoglobulin production). For example, the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

### Variations on Methods

The disclosed methods detect the presence or determine the amount or level of at least one type of anti-SARS-CoV-2 antibody and at least one type of SARS-CoV-2 antigen present in a biological sample as described herein. The methods may also be adapted in view of other methods for analyzing analytes. Examples of well-known variations include, but are not limited to, immunoassay, competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, single molecule detection assay, lateral flow assay, etc.

Immunoassay. The analyte of interest, such as the at least one type of anti-SARS-CoV-2 antibody and at least one type of SARS-CoV-2 antigen, as described above, may be analyzed using the specific binding partners, as described above, in an immunoassay. The presence or amount of the analyte (e.g., at least one type of anti-SARS-CoV-2 antibody and at least one type of SARS-CoV-2 antigen) present in a biological sample may be readily determined using an immunoassay. For example, in one aspect, one method that can be used is a chemiluminescent microparticle immunoassay, in particular one employing the ARCHITECT® automated analyzer (Abbott Laboratories, Abbott Park, IL). Other methods that can be used include, for example, mass spectrometry, and immunohistochemistry (e.g., with sections from tissue biopsies). Additionally, methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of an antibody to an analyte (e.g., a SARS-CoV-2 antigen) can be detected via direct labels, such as fluorescent or luminescent tags, metals and radionuclides attached to the antibody or via indirect labels, such as alkaline phosphatase or horseradish peroxidase.

The at least one first specific binding partner, at least one second specific binding partner, at least one third specific binding partner, and at least one fourth specific binding partner are immobilized on a microparticle. In some embodiments, the at least one fifth specific binding partner, at least one sixth specific binding partner, at least one seventh specific binding partner, and at least one eighth specific binding partner may also be incorporated onto a solid support. The at least one fifth specific binding partner, at least one sixth specific binding partner, at least one seventh specific binding partner, and at least one eighth specific binding partner specific binding partners may be immobilized onto a variety of supports, such as magnetic or chromatographic matrix particles, the surface of an assay plate (such as microtiter wells), pieces of a solid substrate material, and the like. An assay strip can be prepared by coating the antigen and/or antibody or plurality of antibodies in an array on a solid support. This strip can then be dipped into the test sample and processed quickly through washes and detection steps to generate a measurable signal, such as a colored spot.

A homogeneous format may be used. For example, after the biological sample is obtained from a subject, a mixture is prepared. The mixture contains the test sample being assessed for the analyte (e.g., of at least one type of anti-SARS-CoV-2 antibody and at least one type of SARS-CoV-2 antigen), at least one capture composition and at least one detection composition. The order in which the test sample, the at least one capture composition, and the at least one detection composition are added to form the mixture is not critical. In some embodiments, the test sample is simultaneously contacted with the at least one capture composition and the at least one detection composition.

A heterogeneous format may be used. For example, after the biological sample is obtained from a subject, a first mixture is prepared. The mixture contains the biological sample being assessed for the analyte (e.g., of at least one type of anti-SARS-CoV-2 antibody and at least one type of SARS-CoV-2 antigen) and at least one capture composition. The order in which the biological sample and the at least one capture composition are added to form the mixture is not critical.

The at least one capture composition may comprise at least two different types of microparticle reagents comprising a first microparticle reagent that specifically binds to at least one type of SARS-CoV-2 antigen, and a second microparticle reagent that specifically binds to at least one type of anti-SARS-CoV-2 antibody or antibody fragment or variant thereof. The microparticle may be a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO—Fe₂O₃). Beads can have a solid core portion that is magnetic and is surrounded by one or more non-magnetic layers. Alternately, the magnetic portion can be a layer around a non-magnetic core. The solid support on which the first specific binding partner is immobilized may be stored in dry form or in a liquid. The magnetic beads may be subjected to a magnetic field prior to or after contacting with the sample with a magnetic bead on which the first specific binding partner is immobilized.

After the mixture containing the first or second microparticle reagent-analyte complex is formed, any unbound analyte is removed from the complex using any technique known in the art. For example, the unbound analyte can be removed by washing. Desirably, in some instances, the first and/or second microparticle reagent is present in excess of any analyte present in the test sample, such that all or most analyte that is present in the test sample is bound by the first specific binding partner.

After any unbound analyte is removed, at least one detection composition is added to the mixture to form a first or second microparticle reagent-analyte complex-first or second detection reagent complex. Moreover, the first or second detection reagent is labeled with or contains a detectable label as described above.

One-Step Immunoassay or “Capture on the Fly” Assay. In a capture on the fly immunoassay, a solid substrate is pre-coated with an immobilization agent. The capture composition, the analyte and the detection composition are added to the solid substrate together, followed by a wash step prior to detection. The capture composition can bind the analyte and comprises a ligand or property that interacts with the immobilization agent. The capture composition and the detection composition may comprise any moiety capable of capture or detection as described herein or known in the art.

In certain other embodiments, in a one-step immunoassay or “capture on the fly,” the capture composition comprises at least two different types of microparticle reagents pre-coated with an immobilization agent (such as biotin, streptavidin, etc.) and at least one of a first specific binding partner, second specific binding partner, third specific binding partner, and/or a fourth specific binding partner. The first specific binding partner, second specific binding partner, third specific binding partner, and/or a fourth specific binding partner binds to the analyte of interest. The detection composition comprises a detection reagent with a detectable label which binds to an analyte of interest.

The solid support and the capture and detection compositions may be added to a test sample (either sequentially or simultaneously). The ligand on the at least two different types of microparticle reagents binds to the immobilization agent on the solid support to form a solid support/microparticle reagent complex. Any analyte of interest present in the sample binds to the solid support/microparticle reagent complex to form a solid support/microparticle reagent/analyte complex. The detection reagent binds to the solid support/microparticle reagent/analyte complex and the detectable label is detected. An optional wash step may be employed before the detection. In certain embodiments, in a one-step assay more than one analyte may be measured.

The use of a capture on the fly assay can be done in a variety of formats as described herein, and known in the art. For example, the format can be a sandwich assay such as described above, but alternately can be a competition assay, can employ any number of microparticle reagents, or use other variations such as are known.

Forward Competitive Inhibition Assay. In a forward competitive format, an aliquot of labeled analyte of interest (e.g., at least one type of anti-SARS-CoV-2 antibody and at least one type of SARS-CoV-2 antigen) having a fluorescent label, a tag attached with a cleavable linker, etc.) of a known concentration is used to compete with analyte of interest (e.g., at least one type of anti-SARS-CoV-2 antibody and at least one type of SARS-CoV-2 antigen) in a biological sample for binding to antibody directed against analyte of interest.

In a forward competition assay, a microparticle reagent can either be sequentially or simultaneously contacted with the biological sample and a labeled analyte of interest, labeled analyte of interest fragment or labeled analyte of interest variant thereof. The analyte of interest, analyte of interest fragment or analyte of interest variant, can be labeled with any detectable label, including a detectable label comprised of tag attached with a cleavable linker.

The labeled analyte of interest, the biological sample and the microparticle reagent are incubated at a pH of from about 4.5 to about 10.0, at a temperature of from about 2° C. to about 45° C., and for a period from at least one (1) minute to about eighteen (18) hours, from about 2-6 minutes, from about 7-12 minutes, from about 5-15 minutes, or from about 3-4 minutes. Two different species of complexes may then be generated. Specifically, one of the microparticle reagent and/or recombinant antigen-analyte of interest complexes generated contains a detectable label (e.g., a fluorescent label, etc.) while the other microparticle reagent and/or recombinant antigen-analyte of interest complex does not contain a detectable label. The microparticle reagent and/or recombinant antigen-analyte of interest complex can be, but does not have to be, separated from the remainder of the biological sample prior to quantification of the detectable label. Regardless of whether the microparticle reagent and/or recombinant antigen-analyte of interest complex is separated from the remainder of the biological sample, the amount of detectable label in the antibody and/or recombinant antigen-analyte of interest complex is then quantified. The concentration of analyte of interest in the biological sample can then be determined.

Reverse Competitive Inhibition Assay. Similar to the forward competitive format, a reverse competition assay, an immobilized analyte of interest can either be sequentially or simultaneously contacted with a test sample and at least one labeled recombinant specific binding partner. The analyte of interest can be bound to a solid support, such as the solid supports discussed above.

The immobilized analyte of interest, biological sample and at least one analyte specific binding partner are incubated under conditions similar to those described above. Two different types of complexes are then generated. Specifically, one of the analyte of interest-specific binding partner complexes generated is immobilized and contains a detectable label (e.g., a fluorescent label, etc.) while the other analyte of interest-specific binding partner complex is not immobilized and contains a detectable label. The non-immobilized analyte of interest-specific binding partner complex and the remainder of the biological sample are removed from the presence of the immobilized analyte of interest-specific binding partner complex through techniques known in the art, such as washing. Once the non-immobilized analyte of interest-specific binding partner complex is removed, the amount of detectable label in the immobilized analyte of interest-specific binding partner complex is then quantified. The concentration of analyte of interest in the test sample can then be determined by comparing the quantity of detectable label.

Single Molecule Detection Assay. Single molecule detection assays and methods, such as the use of a nanopore device or nanowell device, also can be used. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety. Other devices and methods appropriate for single molecule detection also can be employed.

Lateral Flow Assays. Lateral flow assays are generally provided in a device comprising a lateral flow test strip (e.g., nitrocellulose or filter paper), a sample application area (e.g., sample pad), a test results area (e.g., a test line), an optional control results area (e.g., a control line), and an analyte-specific binding partner that is bound to a detectable label (e.g., a colored particle or an enzyme detection system). See, e.g., U.S. Pat. Nos. 6,485,982; 6,187,598; 5,622,871; 6,565,808; and 6,809,687; and U.S. patent application Ser. No. 10/717,082, each of which is incorporated herein by reference.

In some embodiments, the present disclosure provides assays for detecting at least one type of anti-SARS-CoV-2 antibody and at least one type of SARS-CoV-2 antigen in a sample. In some embodiments, the technology relates to analytical devices that are suitable for use in the home, clinic, or hospital, and that are intended to give an analytical result that is rapid with minimum degree of skill and involvement from the user. In some embodiments, use of the devices described herein involves methods in which a user performs a sequence of operations to provide an observable test result.

In some embodiments, also provided is a test device comprising a reagent-impregnated test strip to provide a specific binding assay, e.g., an immunoassay. In some embodiments, a sample is applied to one portion of the test strip and is allowed to permeate through the strip material, usually with the aid of an eluting solvent such as water and/or a suitable buffer (e.g., an extraction buffer optionally comprising a detergent). In so doing, the sample progresses into or through a detection zone in the test strip wherein the analyte suspected of being in the sample is immobilized. Analyte present in the sample can therefore become bound within the detection zone. The extent to which the analyte becomes bound in that zone can be determined with the aid of labelled reagents that also can be incorporated in the test strip or applied thereto subsequently.

In some embodiments, the analytical test device comprises a hollow casing constructed of moisture-impervious solid material containing a dry porous carrier that communicates directly or indirectly with the exterior of the casing such that a liquid test sample can be applied to the porous carrier. Another aspect relates to a device that comprises a porous solid phase material carrying in a first zone the detection reagent that is retained in the first zone while the porous material is in the dry state but is free to migrate through the porous material when the porous material is moistened, for example, by the application of an aqueous liquid sample suspected of containing the analyte. In some embodiments, the porous material comprises in a second zone, which is spatially distinct from the first zone, comprising the microparticle reagents having specificity for the analyte and which is capable of participating with the detection reagent in either a “sandwich” or a “competition” reaction. The microparticle reagents is firmly immobilized on the porous material such that it is not free to migrate when the porous material is in the moist state. In some embodiments, a device as described herein is contacted with an aqueous liquid sample suspected of containing the analyte, such that the sample permeates by capillary action through the porous solid phase material via the first zone into the second zone and the labelled reagent migrates therewith from the first zone to the second zone, the presence of analyte in the sample being determined.

Examples of lateral flow assays that can be used in the present disclosure include, for example, PANBIO®, BINAX® and BINAXNOW® (Alere, Abbott Park, IL).

### Samples and Controls

Test or Biological Sample. As used herein, the terms “sample,” “test sample,” and “biological sample” refer to a fluid sample containing or suspected of containing at least one type of anti-SARS-CoV-2 antibody and at least one type of SARS-CoV-2 antigen. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing at least one type of anti-SARS-CoV-2 antibody and/or at least one type of SARS-CoV-2 antigen may be assayed directly. In a particular example, the source of at least one type of anti-SARS-CoV-2 antibody and/or at least one type of SARS-CoV-2 antigen is a mammalian (e.g., human) bodily substance (e.g., a tissue) or bodily fluid. Examples of suitable bodily substances or fluids include, but are not limited to, whole blood (including, for example, capillary blood, venous blood, etc.), serum, plasma, urine, saliva, sweat, sputum, semen, mucus, lacrimal fluid, lymph fluid, amniotic fluid, interstitial fluid, anal swab specimens, nasal mucus specimens, lower respiratory specimens (e.g., endotracheal aspirate or bronchoalveolar lavage), cerebrospinal fluid, feces, one or more dried blood spots, etc. Tissues may include, but are not limited to, oropharyngeal specimens, nasopharyngeal specimens, skeletal muscle tissue, liver tissue, lung tissue, kidney tissue, myocardial tissue, brain tissue, bone marrow, cervix tissue, skin, etc. The sample may be a liquid extract of a solid sample. In certain cases, the source of the sample may be an organ or tissue, such as a biopsy sample, which may be solubilized by tissue disintegration/cell lysis. Additionally, the sample can be a nasopharyngeal or oropharyngeal sample obtained using one or more swabs that, once obtained, is placed in a sterile tube containing a virus transport media (VTM) or universal transport media (UTM), for testing.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 L, or between about 0.1 μL and about 10 μL.

As discussed above, a fluid sample may be diluted prior to use in an assay. For example, in embodiments where the source of the at least one type of anti-SARS-CoV-2 antibody and the at least one type of SARS-CoV-2 antigen is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, a fluid sample is not diluted prior to use in an assay. In some embodiments, the diluent may optionally contain an antibody, such as an IgG antibody that is added to remove any IgG antibodies from the sample.

In some cases, the sample may undergo pre-analytical processing or pre-treatment. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art.

In some cases, pre-treatment may involve adding an antibody, such as an IgG and/or IgM antibody to the biological sample prior to the addition of the at least one capture compositions.

In some cases, the fluid sample may be concentrated prior to use in an assay. For example, in embodiments where the source of the at least one type of anti-SARS-CoV-2 antibody and the at least one type of SARS-CoV-2 antigen is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

Controls and Calibrators. It may be desirable to include a control (such as a positive and/or negative control, which are well known in the art). For example, a positive control can be purified from in vivo or any recombinant SARS-CoV-2 antigen (e.g., nucleocapsid protein) or variant thereof that binds to the first specific binding partner (e.g., anti-SARS-CoV antibody, anti-SARS-CoV-2 antibody or any antibody fragment or variant thereof). In some embodiments, the positive control can be a full-length SARS-CoV-2 antigen (such as a human SARS-CoV-2 nucleocapsid or spike protein). Alternatively, the positive control can be a fragment or variant of the full-length SARS-CoV-2 antigen (such as a human SARS-CoV-2 nucleocapsid or spike protein (e.g., RBD of SARS-CoV-2 spike protein). In some embodiments, a control or calibrator can be a SARS-CoV-2 antigen that is produced recombinantly, using methods described herein and as known in the art. In some embodiments, the control can be a cell culture derived virus (that may or may not have been purified, e.g., lysates or cell culture medium). Examples of come cell culture derived viruses that can be used include Vero cells that have been infected with SARS-CoV-2.

Alternatively, or in addition, a positive control can be at least one type of anti-SARS-CoV-2 antibody or any fragment or variant thereof that binds to at least one type of SARS-CoV-2 antigen any fragment or variant thereof). In some embodiments, the positive control can be an anti-SARS-CoV-2 IgA antibody, an anti-SARS-CoV-2 IgM antibody, an anti-SARS-CoV-2 IgG antibody directed to a full-length SARS-CoV-2 antigen or a fragment or variant thereof. In some embodiments, a control or calibrator can be an anti-SARS-CoV-2 antibody that is produced recombinantly, using methods described herein and as known in the art.

The control may be analyzed separately from, or concurrently with, the sample from the subject as described above. The results obtained from the subject sample can be compared to the results or information obtained from the control sample. Standard curves may be provided or developed with use of the calibrators and controls, with which assay results for the sample may be compared. Such standard curves typically present levels of marker as a function of assay units (i.e., fluorescent signal intensity, if a fluorescent label is used).

It may also be desirable to include one or more calibrators for use in calibrating of any automated or semi-automated system for which the methods and kits described herein are adapted for use. The use of calibrators in such systems is well known in the art. For example, one or more calibrators can include the full-length SARS-CoV-2 antigen (such as a human SARS-CoV-2 nucleocapsid protein) or an isolated anti-SARS-CoV-2 antibody. Alternatively, the calibrator can be a fragment or variant of the full-length SARS-CoV-2 nucleocapsid antigen or the anti-SARS-CoV-2 antibody.

In some embodiments, the calibrator can be a cell culture derived virus (that may or may not have been purified, e.g., lysates or cell culture medium). Examples of cell culture derived viruses that can be used include Vero cells that have been infected with SARS-CoV or SARS-CoV-2. In some embodiments, the calibrator can be a clinical sample or an isolate from a clinical sample from a subject known to have at least one type of SARS-CoV-2 antigen and/or at least one type of anti-SARS-CoV-2 antibody.

The calibrator may optionally be part of a series of calibrators in which each of the calibrators differs from the other calibrators in the series by the concentration of at least one type of SARS-CoV-2 antigen or at least one type of anti-SARS-CoV-2 antibody.

### Kit

Provided herein is a kit, which may be used in the methods described herein for assaying or assessing a test sample for at least one type of SARS-CoV-2 antigen or at least one type of anti-SARS-CoV-2 antibody. The kit comprises at least one component for assaying the test sample for at least one type of SARS-CoV-2 antigen and/or at least one type of anti-SARS-CoV-2 antibody, as well as instructions for assaying the test sample for at least one type of SARS-CoV-2 antigen or at least one type of anti-SARS-CoV-2 antibody. For example, the kit can comprise instructions for assaying the test sample for at least one type of SARS-CoV-2 antigen and at least one type of anti-SARS-CoV-2 antibody using an immunoassay, e.g., chemiluminescent microparticle immunoassay. Instructions included in kits can be affixed to packaging material, can be included as a package insert, or can be viewed or downloaded from a particular website that is recited as part of the kit packaging or inserted materials. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component for assaying the test sample for at least one type of anti-SARS-CoV-2 antibody and at least one type of SARS-CoV-2 antigen may include at least capture composition and/o rat least one detection composition as described previously herein. The kit further can include at least one type of anti-SARS-CoV-2 antigen or at least one type of anti-SARS-CoV-2 antibody purified from in vivo or recombinant, for use as calibrators or controls, or optionally, these can be provided separately.

Alternatively, or additionally, the kit can comprise a calibrator or control, e.g., purified, and optionally frozen or lyophilized, as described previously herein, and/or at least one container (e.g., tubes, microtiter plates or strips) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

The kit may further comprise reference standards for quantifying the at least one type of SARS-CoV-2 antigen and the at least one type of anti-SARS-CoV-2 antibody. The reference standards may be employed to establish standard curves for interpolation and/or extrapolation of the at least one type of SARS-CoV-2 antigen concentration and/or the at least one type of anti-SARS-CoV-2 antibody concentration.

Any antibodies, such as recombinant antibodies and/or any recombinant antigens, which are provided in the kit, can incorporate a detectable label, such as a fluorophore, radioactive moiety, enzyme, biotin/avidin label, chromophore, chemiluminescent label, or the like, or the kit can include reagents for labeling the components of the kit or the analytes (e.g., the SARS-CoV-2 antigen and the anti-SARS-CoV-2 antibody).

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays,

The kit also can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents or extraction buffers), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., tubes and microtiter plates. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit also can include one or more instrument for assisting with obtaining a test sample, such as a syringe, pipette, forceps, measured spoon, or the like.

The kit also can include one or more sample collection/acquisition instruments for assisting with obtaining a test sample (e.g., microsampling devices, micro-needles, or other minimally invasive pain-free blood collection methods; blood collection tube(s); lancets; capillary blood collection tubes; other single fingertip-prick blood collection methods; buccal swabs, nasal/throat swabs; 16-gauge or other size needle, surgical knife or laser (e.g., particularly hand-held), syringes, sterile container, or cannula, for obtaining, storing, or aspirating tissue samples).

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, including for example biomarkers or markers for other infectious agents.

### Adaptation of Kit and Method

The kit (or components thereof), as well as the method for detecting the presence or determining the amount or level or concentration of at least one type of SARS-CoV-2 antigen and at least one type of anti-SARS-CoV-2 antibody in a test sample by an immunoassay as described herein, can be adapted for use in a variety of automated and semi-automated systems or platforms (including those wherein the solid phase comprises a microparticle), as described in, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, IL) as Abbott Point of Care (i-STAT® or i-STAT Alinity, Abbott Laboratories) as well as those described in U.S. Pat. Nos. 5,089,424 and 5,006,309, and as commercially marketed, e.g., by Abbott Laboratories (Abbott Park, IL) as ARCHITECT® or the series of Abbott Alinity devices. Such systems include one or more devices and/or components that can be used to detect one or more labels in the resulting complexes formed in the methods described previously herein.

Some of the differences between an automated or semi-automated system as compared to a non-automated system include the substrate to which the first specific binding partner (e.g., recombinant antigen or capture reagent) is attached, and the length and timing of the capture, detection, and/or any optional wash steps. Whereas a non-automated format may require a relatively longer incubation time with test sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories that may be used include, but are not limited to, AXSYM®, IMX® (see, e.g., U.S. Pat. No. 5,294,404, which is hereby incorporated by reference in its entirety), PRISM®, EIA (bead), and QUANTUM™ II, as well as other platforms. Additionally, the assays, kits, and kit components can be employed in other formats, for example, on electrochemical or other hand-held or point-of-care assay systems. As mentioned previously, the present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described in, for example, U.S. Pat. No. 5,063,081, U.S. Patent App. Publication Nos. 2003/0170881, 2004/0018577, 2005/0054078, and 2006/0160164, which are incorporated by reference herein in their entireties.

The methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, IL) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, IL), which comprises a buffer containing MES, other salt, a protein blocker, and an antimicrobial agent. Additionally, as described in U.S. Patent Application No. 61/142,048, improved signal generation may be obtained, e.g., in an i-STAT® cartridge format, using a nucleic acid sequence linked to the signal antibody as a signal amplifier.

### Analysis and Interpretation of Results

The results obtained using the methods of the present disclosure (e.g., detecting the presence of at least one type of SARS-CoV-2 antigen and at least one type of anti-SARS-CoV-2 antibody or determining the amount, level or concentration of at least one type of SARS-CoV-2 antigen and at least one type of anti-SARS-CoV-2 antibody in a biological sample) can be analyzed and interpreted individually or in combination with other any other results obtained prior to, during or after the results of the methods of the present disclosure are performed. The nature of the other results analyzed and interpreted with the results of the present disclosure are changeable.

The present disclosure has multiple embodiments, illustrated by the following non-limited examples.

It will be readily apparent to those skilled in the art that other suitable modifications and adaptations of the methods of the present disclosure described herein are readily applicable and appreciable, and may be made using suitable equivalents without departing from the scope of the present disclosure or the embodiments and embodiments disclosed herein. Having now described the present disclosure in detail, the same will be more clearly understood by reference to the following examples, which are merely intended only to illustrate some embodiments and embodiments of the disclosure, and should not be viewed as limiting to the scope of the disclosure. The disclosures of all journal references, U.S. patents, and publications referred to herein are hereby incorporated by reference in their entireties.

For reasons of completeness, various aspects are set out in the following numbered clauses:

Clause 1. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof;
at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex;
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample.

Clause 2. The method of clause 1, wherein the first microparticle reagent and the second microparticle reagent comprise at least one microparticle.

Clause 3. The method of clause 1 or clause 2, wherein the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof.

Clause 4. The method of any of clauses 1-3, wherein the second microparticle reagent comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof.

Clause 5. The method of clause 3 or clause 4, wherein the first detection reagent further comprises:
(i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner,
thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof.

Clause 6. The method of clause 4 or clause 5, wherein the second detection reagent further comprises:
(i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner,
thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

Clause 7. The method of clause 6, wherein the signal from the (1) the first complex indicates the presence or amount of anti-SARS-CoV-2 spike RBD antigen or fragment or variant thereof in the sample; (2) the second complex indicates the presence or amount of anti-SARS-CoV-2 nucleocapsid antigen or fragment or variant thereof in the sample; (3) the third complex indicates the presence or amount of anti-SARS-CoV-2 spike RBD antibody or antibody fragment or variant thereof in the sample; and (4) the fourth complex indicates the presence or amount of anti-SARS-CoV-2 nucleocapsid antibody or antibody fragment or variant thereof in the sample.

Clause 8. The method of any of clauses 1-7, wherein the biological sample is whole blood, serum, plasma, saliva, a nasal mucus specimen, an anal swab specimen, an oropharyngeal specimen, or a nasopharyngeal specimen.

Clause 9. The method of any of clauses 4-8, wherein the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

Clause 10. The method of any of clauses 1-9, wherein the at least one type of anti-SARS-CoV-2 antibody detected is an anti-SARS-CoV-2 IgA antibody, an anti-SARS-CoV-2 IgM antibody, an anti-SARS-CoV-2 IgG antibody or any combination thereof.

Clause 11. The method of any of clauses 1-9, wherein none of the at least two different types of microparticle reagents, at least one first detection reagent, and the at least one second detection reagent include any anti-species antibodies.

Clause 12. The method of any of clauses 1-9, wherein the at least two different types of microparticle reagents, the at least one of the first detection reagent, the at least one second detection reagent or both the at least one first detection reagent and at least one second detection reagent is an anti-species IgA (e.g., anti-human-IgA IgG) antibody, an anti-species IgG (e.g., anti-human-IgG IgG) antibody, an anti-species IgM (e.g., anti-human-IgM IgG) antibody, or any combination thereof.

Clause 13. The method of any of clauses 1-12, wherein the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 and monitoring the subject for SARS-CoV-2 IgA, SARS-CoV-2 IgG, SARS-CoV-2 IgM antibodies and/or at least one type of SARS-CoV-2 antigen.

Clause 14. The method of clause 13, wherein SARS-CoV-2 infection is detected by determining the presence of SARS-CoV-2 viral RNA using polymerase chain reaction, or by determining the presence of a SARS-CoV-2 viral antigen.

Clause 15. The method of any of clauses 1-14, wherein the method is performed in from about 5 to about 20 minutes, and optionally is performed in about 15 to 30 minutes.

Clause 16. The method of any of clauses 1-15, wherein the method further comprises use with at least one calibrator reagent, at least one control reagent, or at least one calibrator reagent and at least one control reagent.

Clause 17. The method of any of clauses 1-16, wherein the method is selected from the group consisting of an immunoassay or a clinical chemistry assay.

Clause 18. The method of any of clauses 1-17, wherein the method is performed using single molecule detection, a lateral flow assay, or a point-of-care assay.

Clause 19. The method of any of clauses 1-18, wherein the method is adapted for use in an automated system or a semi-automated system.

Clause 20. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof;
at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex;
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample, wherein the first microparticle reagent and the second microparticle reagent comprise at least one microparticle.

Clause 21. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
   at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof;
   at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex;
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample,
wherein the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof.

Clause 22. The method of clause 21, wherein the first microparticle reagent and the second microparticle reagent comprise at least one microparticle.

Clause 23. The method of clause 21 or clause 22, wherein the second microparticle reagent comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof.

Clause 24. The method of any of clauses 21-23, wherein the first detection reagent further comprises:
(i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner,
thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof.

Clause 25. The method of any of clauses 21-24, wherein the second detection reagent further comprises:
(i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner,
thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

Clause 26. The method of clause 25, wherein the signal from the (1) the first complex indicates the presence or amount of anti-SARS-CoV-2 spike RBD antigen or fragment or variant thereof in the sample; (2) the second complex indicates the presence or amount of anti-SARS-CoV-2 nucleocapsid antigen or fragment or variant thereof in the sample; (3) the third complex indicates the presence or amount of anti-SARS-CoV-2 spike RBD antibody or antibody fragment or variant thereof in the sample; and (4) the fourth complex indicates the presence or amount of anti-SARS-CoV-2 nucleocapsid antibody or antibody fragment or variant thereof in the sample.

Clause 27. The method of any of clauses 21-26, wherein the biological sample is whole blood, serum, plasma, saliva, a nasal mucus specimen, an anal swab specimen, an oropharyngeal specimen, or a nasopharyngeal specimen.

Clause 28. The method of any of clauses 23-27, wherein the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

Clause 29. The method of any of clauses 21-29, wherein the at least one type of anti-SARS-CoV-2 antibody detected is an anti-SARS-CoV-2 IgA antibody, an anti-SARS-CoV-2 IgM antibody, an anti-SARS-CoV-2 IgG antibody or any combination thereof.

Clause 30. The method of any of clauses 21-29, wherein none of the at least two different types of microparticle reagents, at least one first detection reagent, and the at least one second detection reagent include any anti-species antibodies.

Clause 31. The method of any of clauses 21-29, wherein the at least two different types of microparticle reagents, the at least one of the first detection reagent, the at least one second detection reagent or both the at least one first detection reagent and at least one second detection reagent is an anti-species IgA (e.g., anti-human-IgA IgG) antibody, an anti-species IgG (e.g., anti-human-IgG IgG) antibody, an anti-species IgM (e.g., anti-human-IgM IgG) antibody, or any combination thereof.

Clause 32. The method of any of clauses 21-31, wherein the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 and monitoring the subject for SARS-CoV-2 IgA, SARS-CoV-2 IgG, SARS-CoV-2 IgM antibodies and/or at least one type of SARS-CoV-2 antigen.

Clause 33. The method of clause 32, wherein SARS-CoV-2 infection is detected by determining the presence of SARS-CoV-2 viral RNA using polymerase chain reaction, or by determining the presence of a SARS-CoV-2 viral antigen.

Clause 34. The method of any of clauses 21-33, wherein the method is performed in from about 5 to about 20 minutes, and optionally is performed in about 15 to 30 minutes.

Clause 35. The method of any of clauses 21-34, wherein the method further comprises use with at least one calibrator reagent, at least one control reagent, or at least one calibrator reagent and at least one control reagent.

Clause 36. The method of any of clauses 21-35, wherein the method is selected from the group consisting of an immunoassay or a clinical chemistry assay.

Clause 37. The method of any of clauses 21-36, wherein the method is performed using single molecule detection, a lateral flow assay, or a point-of-care assay.

Clause 38. The method of any of clauses 21-37, wherein the method is adapted for use in an automated system or a semi-automated system.

Clause 39. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
   at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof;
   at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex;
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample,
wherein the second microparticle reagent comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof.

Clause 40. The method of clause 39, wherein the first microparticle reagent and the second microparticle reagent comprise at least one microparticle.

Clause 41. The method of clause 39 or clause 40, wherein the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof.

Clause 42. The method of any of clauses 39-41, wherein the first detection reagent further comprises:
(i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner,
thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof.

Clause 43. The method of any of clauses 39-42, wherein the second detection reagent further comprises:
(i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner,
thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

Clause 44. The method of clause 43, wherein the signal from the (1) the first complex indicates the presence or amount of anti-SARS-CoV-2 spike RBD antigen or fragment or variant thereof in the sample; (2) the second complex indicates the presence or amount of anti-SARS-CoV-2 nucleocapsid antigen or fragment or variant thereof in the sample; (3) the third complex indicates the presence or amount of anti-SARS-CoV-2 spike RBD antibody or antibody fragment or variant thereof in the sample; and (4) the fourth complex indicates the presence or amount of anti-SARS-CoV-2 nucleocapsid antibody or antibody fragment or variant thereof in the sample.

Clause 45. The method of any of clauses 39-44, wherein the biological sample is whole blood, serum, plasma, saliva, a nasal mucus specimen, an anal swab specimen, an oropharyngeal specimen, or a nasopharyngeal specimen.

Clause 46. The method of any of clauses 39-45, wherein the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

Clause 47. The method of any of clauses 39-46, wherein the at least one type of anti-SARS-CoV-2 antibody detected is an anti-SARS-CoV-2 IgA antibody, an anti-SARS-CoV-2 IgM antibody, an anti-SARS-CoV-2 IgG antibody or any combination thereof.

Clause 48. The method of any of clauses 39-46, wherein none of the at least two different types of microparticle reagents, at least one first detection reagent, and the at least one second detection reagent include any anti-species antibodies.

Clause 49. The method of any of clauses 39-46, wherein the at least two different types of microparticle reagents, the at least one of the first detection reagent, the at least one second detection reagent or both the at least one first detection reagent and at least one second detection reagent is an anti-species IgA (e.g., anti-human-IgA IgG) antibody, an anti-species IgG (e.g., anti-human-IgG IgG) antibody, an anti-species IgM (e.g., anti-human-IgM IgG) antibody, or any combination thereof.

Clause 50. The method of any of clauses 39-49, wherein the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 and monitoring the subject for SARS-CoV-2 IgA, SARS-CoV-2 IgG, SARS-CoV-2 IgM antibodies and/or at least one type of SARS-CoV-2 antigen.

Clause 51. The method of clause 50, wherein SARS-CoV-2 infection is detected by determining the presence of SARS-CoV-2 viral RNA using polymerase chain reaction, or by determining the presence of a SARS-CoV-2 viral antigen.

Clause 52. The method of any of clauses 39-51, wherein the method is performed in from about 5 to about 20 minutes, and optionally is performed in about 15 to 30 minutes.

Clause 53. The method of any of clauses 39-52, wherein the method further comprises use with at least one calibrator reagent, at least one control reagent, or at least one calibrator reagent and at least one control reagent.

Clause 54. The method of any of clauses 39-53, wherein the method is selected from the group consisting of an immunoassay or a clinical chemistry assay.

Clause 55. The method of any of clauses 39-54, wherein the method is performed using single molecule detection, a lateral flow assay, or a point-of-care assay.

Clause 56. The method of any of clauses 39-55, wherein the method is adapted for use in an automated system or a semi-automated system.

Clause 57. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
   at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof;
   at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex;
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample,
wherein the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof, and further
wherein the first detection reagent further comprises:
(i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner,
thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof.

Clause 58. The method of clause 57, wherein the first microparticle reagent and the second microparticle reagent comprise at least one microparticle.

Clause 59. The method of clause 57 or clause 58, wherein the second microparticle reagent comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof.

Clause 60. The method of any of clauses 57-59, wherein the second detection reagent further comprises:
(i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner,
thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

Clause 61. The method of clause 60, wherein the signal from the (1) the first complex indicates the presence or amount of anti-SARS-CoV-2 spike RBD antigen or fragment or variant thereof in the sample; (2) the second complex indicates the presence or amount of anti-SARS-CoV-2 nucleocapsid antigen or fragment or variant thereof in the sample; (3) the third complex indicates the presence or amount of anti-SARS-CoV-2 spike RBD antibody or antibody fragment or variant thereof in the sample; and (4) the fourth complex indicates the presence or amount of anti-SARS-CoV-2 nucleocapsid antibody or antibody fragment or variant thereof in the sample.

Clause 62. The method of any of clauses 57-61, wherein the biological sample is whole blood, serum, plasma, saliva, a nasal mucus specimen, an anal swab specimen, an oropharyngeal specimen, or a nasopharyngeal specimen.

Clause 63. The method of any of clauses 59-62, wherein the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

Clause 64. The method of any of clauses 57-63, wherein the at least one type of anti-SARS-CoV-2 antibody detected is an anti-SARS-CoV-2 IgA antibody, an anti-SARS-CoV-2 IgM antibody, an anti-SARS-CoV-2 IgG antibody or any combination thereof.

Clause 65. The method of any of clauses 57-63, wherein none of the at least two different types of microparticle reagents, at least one first detection reagent, and the at least one second detection reagent include any anti-species antibodies.

Clause 66. The method of any of clauses 57-63, wherein the at least two different types of microparticle reagents, the at least one of the first detection reagent, the at least one second detection reagent or both the at least one first detection reagent and at least one second detection reagent is an anti-species IgA (e.g., anti-human-IgA IgG) antibody, an anti-species IgG (e.g., anti-human-IgG IgG) antibody, an anti-species IgM (e.g., anti-human-IgM IgG) antibody, or any combination thereof.

Clause 67. The method of any of clauses 57-66, wherein the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 and monitoring the subject for SARS-CoV-2 IgA, SARS-CoV-2 IgG, SARS-CoV-2 IgM antibodies and/or at least one type of SARS-CoV-2 antigen.

Clause 68. The method of clause 67, wherein SARS-CoV-2 infection is detected by determining the presence of SARS-CoV-2 viral RNA using polymerase chain reaction, or by determining the presence of a SARS-CoV-2 viral antigen.

Clause 69. The method of any of clauses 57-68, wherein the method is performed in from about 5 to about 20 minutes, and optionally is performed in about 15 to 30 minutes.

Clause 70. The method of any of clauses 57-69, wherein the method further comprises use with at least one calibrator reagent, at least one control reagent, or at least one calibrator reagent and at least one control reagent.

Clause 71. The method of any of clauses 57-70, wherein the method is selected from the group consisting of an immunoassay or a clinical chemistry assay.

Clause 72. The method of any of clauses 57-71, wherein the method is performed using single molecule detection, a lateral flow assay, or a point-of-care assay.

Clause 73. The method of any of clauses 57-72, wherein the method is adapted for use in an automated system or a semi-automated system.

Clause 74. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
   at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof;
   at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex;
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample,
wherein the second microparticle reagent comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof, and further
wherein the second detection reagent further comprises:
(i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner,
thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

Clause 75. The method of clause 74, wherein the first microparticle reagent and the second microparticle reagent comprise at least one microparticle.

Clause 76. The method of clause 73 or clause 74, wherein the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof.

Clause 77. The method of any of claims 74-76, wherein the first detection reagent further comprises:
(i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner,
thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof.

Clause 78. The method of clause 77, wherein the signal from the (1) the first complex indicates the presence or amount of anti-SARS-CoV-2 spike RBD antigen or fragment or variant thereof in the sample; (2) the second complex indicates the presence or amount of anti-SARS-CoV-2 nucleocapsid antigen or fragment or variant thereof in the sample; (3) the third complex indicates the presence or amount of anti-SARS-CoV-2 spike RBD antibody or antibody fragment or variant thereof in the sample; and (4) the fourth complex indicates the presence or amount of anti-SARS-CoV-2 nucleocapsid antibody or antibody fragment or variant thereof in the sample.

Clause 79. The method of any of clauses 74-78, wherein the biological sample is whole blood, serum, plasma, saliva, a nasal mucus specimen, an anal swab specimen, an oropharyngeal specimen, or a nasopharyngeal specimen.

Clause 80. The method of any of clauses 74-78, wherein the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

Clause 81. The method of any of clauses 74-79, wherein the at least one type of anti-SARS-CoV-2 antibody detected is an anti-SARS-CoV-2 IgA antibody, an anti-SARS-CoV-2 IgM antibody, an anti-SARS-CoV-2 IgG antibody or any combination thereof.

Clause 82. The method of any of clauses 74-79, wherein none of the at least two different types of microparticle reagents, at least one first detection reagent, and the at least one second detection reagent include any anti-species antibodies.

Clause 83. The method of any of clauses 74-79, wherein the at least two different types of microparticle reagents, the at least one of the first detection reagent, the at least one second detection reagent or both the at least one first detection reagent and at least one second detection reagent is an anti-species IgA (e.g., anti-human-IgA IgG) antibody, an anti-species IgG (e.g., anti-human-IgG IgG) antibody, an anti-species IgM (e.g., anti-human-IgM IgG) antibody, or any combination thereof.

Clause 84. The method of any of clauses 74-83, wherein the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 and monitoring the subject for SARS-CoV-2 IgA, SARS-CoV-2 IgG, SARS-CoV-2 IgM antibodies and/or at least one type of SARS-CoV-2 antigen.

Clause 85. The method of clause 84, wherein SARS-CoV-2 infection is detected by determining the presence of SARS-CoV-2 viral RNA using polymerase chain reaction, or by determining the presence of a SARS-CoV-2 viral antigen.

Clause 86. The method of any of clauses 74-85, wherein the method is performed in from about 5 to about 20 minutes, and optionally is performed in about 15 to 30 minutes.

Clause 87. The method of any of clauses 74-86, wherein the method further comprises use with at least one calibrator reagent, at least one control reagent, or at least one calibrator reagent and at least one control reagent.

Clause 88. The method of any of clauses 74-87, wherein the method is selected from the group consisting of an immunoassay or a clinical chemistry assay.

Clause 89. The method of any of clauses 74-88, wherein the method is performed using single molecule detection, a lateral flow assay, or a point-of-care assay.

Clause 90. The method of any of clauses 74-89, wherein the method is adapted for use in an automated system or a semi-automated system.

Clause 91. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
   at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof;
   at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex;
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant in the sample,
wherein the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof, further
wherein the first detection reagent further comprises:
(i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner,
thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof, further
wherein the second microparticle reagent comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof, and further
wherein the second detection reagent further comprises:
(i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner,
thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

Clause 92. The method of clause 91, wherein the first microparticle reagent and the second microparticle reagent comprise at least one microparticle.

Clause 93. The method of clause 91 or clause 92, wherein the signal from the (1) the first complex indicates the presence or amount of anti-SARS-CoV-2 spike RBD antigen or fragment or variant thereof in the sample; (2) the second complex indicates the presence or amount of anti-SARS-CoV-2 nucleocapsid antigen or fragment or variant thereof in the sample; (3) the third complex indicates the presence or amount of anti-SARS-CoV-2 spike RBD antibody or antibody fragment or variant thereof in the sample; and (4) the fourth complex indicates the presence or amount of anti-SARS-CoV-2 nucleocapsid antibody or antibody fragment or variant thereof in the sample.

Clause 94. The method of any of clauses 91-93, wherein the biological sample is whole blood, serum, plasma, saliva, a nasal mucus specimen, an anal swab specimen, an oropharyngeal specimen, or a nasopharyngeal specimen.

Clause 95. The method of any of clauses 91-94, wherein the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

Clause 96. The method of any of clauses 91-95, wherein the at least one type of anti-SARS-CoV-2 antibody detected is an anti-SARS-CoV-2 IgA antibody, an anti-SARS-CoV-2 IgM antibody, an anti-SARS-CoV-2 IgG antibody or any combination thereof.

Clause 97. The method of any of clauses 91-95, wherein none of the at least two different types of microparticle reagents, at least one first detection reagent, and the at least one second detection reagent include any anti-species antibodies.

Clause 98. The method of any of clauses 91-95, wherein the at least two different types of microparticle reagents, the at least one of the first detection reagent, the at least one second detection reagent or both the at least one first detection reagent and at least one second detection reagent is an anti-species IgA (e.g., anti-human-IgA IgG) antibody, an anti-species IgG (e.g., anti-human-IgG IgG) antibody, an anti-species IgM (e.g., anti-human-IgM IgG) antibody, or any combination thereof.

Clause 99. The method of any of clauses 91-98, wherein the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 and monitoring the subject for SARS-CoV-2 IgA, SARS-CoV-2 IgG, SARS-CoV-2 IgM antibodies and/or at least one type of SARS-CoV-2 antigen.

Clause 100. The method of clause 99, wherein SARS-CoV-2 infection is detected by determining the presence of SARS-CoV-2 viral RNA using polymerase chain reaction, or by determining the presence of a SARS-CoV-2 viral antigen.

Clause 101. The method of any of clauses 91-100, wherein the method is performed in from about 5 to about 20 minutes, and optionally is performed in about 15 to 30 minutes.

Clause 102. The method of any of clauses 91-101, wherein the method further comprises use with at least one calibrator reagent, at least one control reagent, or at least one calibrator reagent and at least one control reagent.

Clause 103. The method of any of clauses 91-102, wherein the method is selected from the group consisting of an immunoassay or a clinical chemistry assay.

Clause 104. The method of any of clauses 91-103, wherein the method is performed using single molecule detection, a lateral flow assay, or a point-of-care assay.

Clause 105. The method of any of clauses 91-104, wherein the method is adapted for use in an automated system or a semi-automated system.

What is claimed is:

1. A method for detecting a presence or determining an amount of at least one type of anti-SARS-CoV-2 antibody and at least one type of SARS-CoV-2 antigen in a biological sample from a subject, the method comprising the steps of:
a) contacting at least one biological sample, either simultaneously or sequentially, in any order, with
at least one capture composition comprising at least two different types of microparticle reagents, wherein (i) the first microparticle reagent specifically binds to at least one type of SARS-CoV-2 antigen or fragment or variant thereof, and (ii) the second microparticle reagent specifically binds to at least one type of anti-SARS-CoV-2 antibody or antibody fragment or variant thereof, and
at least one detection composition comprising (a) at least one first detection reagent comprising at least one detectable label that specifically binds to the first microparticle reagent to form a first microparticle reagent-first detection reagent complex; and (b) at least one second detection reagent comprising at least one detectable label that specifically binds to the second microparticle reagent to form a second microparticle reagent-second detection reagent complex; and
b) assessing a signal from each of the first microparticle reagent-first detection reagent complex and the second microparticle reagent-second detection reagent complex to indicate the presence or amount of at least one type of anti-SARS-CoV-2 antibody or antibody fragment or variant thereof and at least one type of SARS-CoV-2 antigen or fragment or variant thereof in the sample.

2. The method of claim 1, wherein the first microparticle reagent and the second microparticle reagent comprise at least one microparticle.

3. The method of claim 1, wherein the first microparticle reagent comprises: (i) at least one first specific binding partner comprising an anti-SARS-CoV-2 spike receptor binding domain (RBD) antibody or antibody fragment or variant thereof that specifically binds to at least one SARS-CoV-2 spike RBD antigen or fragment or variant thereof; (ii) at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof; or (iii) at least one first specific binding partner comprising an anti-SARS-CoV-2 spike RBD antibody or antibody fragment or variant thereof that specifically binds to at least one SARS-CoV-2 spike RBD antigen or fragment or variant thereof and at least one second specific binding partner comprising an anti-SARS-CoV-2 nucleocapsid antibody or antibody fragment or variant thereof that specifically binds to at least one SARS-CoV-2 nucleocapsid antigen or fragment or variant thereof.

4. The method of claim 1, wherein the second microparticle reagent-comprises: (i) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof; (ii) at least one fourth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof; or (iii) at least one third specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof and at least one fourth specific binding partner comprising an isolated polypeptide of a nucleocapsid protein from SARS-CoV-2 or a fragment or variant thereof that specifically binds to at one anti-SARS-CoV-2 nucleocapsid antibody or antibody fragment or variant thereof.

5. The method of claim 3, wherein the first detection reagent further comprises:
(i) at least one fifth specific binding partner which comprises an anti-SARS-CoV-2 spike RBD antibody or antibody fragment or variant thereof that specifically binds to at least one SARS-CoV-2 spike RBD antigen or fragment or variant thereof at a different location then the first specific binding partner; (ii) at least one sixth specific binding partner which comprises anti-SARS-CoV-2 nucleocapsid antibody or antibody fragment or variant thereof that specifically binds to at least one SARS-CoV-2 nucleocapsid antigen or fragment or variant thereof at a location different than the first specific binding partner; or (iii) at least one fifth specific binding partner which comprises anti-SARS-CoV-2 receptor spike RBD antibody or antibody fragment or variant thereof that specifically binds to the at least one SARS-CoV-2 spike RDB antigen or fragment or variant thereof at a different location then the first specific binding partner and at least one sixth specific binding partner which comprises an anti-SARS-CoV-2 nucleocapsid antibody or antibody fragment or variant thereof that specifically binds to at least one SARS-CoV-2 nucleocapsid antigen or fragment or variant thereof at a location different than the second specific binding partner,
thereby producing at least one first complex comprising the first specific binding partner-SARS-CoV-2-spike RBD antigen-fifth specific binding partner and a detectable label, at least one second complex comprising the second specific binding partner-SARS-CoV-2 nucleocapsid antigen-sixth specific binding partner and a detectable label, or a combination thereof.

6. The method of claim 4, wherein the second detection reagent further comprises:
(i) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner; (ii) at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant thereof at a different location then the at least one fourth specific binding partner; or (iii) at least one seventh specific binding partner comprising an isolated polypeptide comprising a RBD of a spike protein from SARS-CoV-2 or a fragment or variant thereof that specifically binds to at least one anti-SARS-CoV-2 spike RBD antibody or antibody fragment or variant thereof at a different location then the at least one third specific binding partner and at least one eighth specific binding partner comprising an isolated polypeptide of a nucleocapsid protein from SARS-CoV-2 or a fragment or variant thereof that specifically binds to at least one anti-SARS-CoV-2 nucleocapsid antibody or antibody fragment or variant at a different location then the at least one fourth specific binding partner,
thereby producing at least one third complex comprising the third specific binding partner-anti-SARS-CoV-2-spike RBD antibody-seventh specific binding partner and a detectable label, at least one fourth complex comprising the fourth specific binding-anti-SARS-CoV-2 nucleocapsid antibody-eighth specific binding partner and a detectable label, or a combination thereof.

7. The method of claim 6, wherein the signal from the (1) the first complex indicates the presence or amount of anti-SARS-CoV-2 spike RBD antigen or fragment or variant thereof in the sample; (2) the second complex indicates the presence or amount of anti-SARS-CoV-2 nucleocapsid antigen or fragment or variant thereof in the sample; (3) the third complex indicates the presence or amount of anti-SARS-CoV-2 spike RBD antibody or antibody fragment or variant in the sample; and (4) the fourth complex indicates the presence or amount of anti-SARS-CoV-2 nucleocapsid antibody or antibody fragment or variant in the sample.

8. The method of claim 1, wherein the biological sample is whole blood, serum, plasma, saliva, a nasal mucus specimen, an anal swab specimen, an oropharyngeal specimen, or a nasopharyngeal specimen.

9. The method of claim 4, wherein the isolated polypeptide of a nucleocapsid protein from SARS-CoV-2 or a fragment or variant thereof that specifically binds to at least one anti-SARS-CoV-2 nucleocapsid antibody or antibody fragment or variant thereof comprises the C-terminal domain nucleocapsid protein from SARS-CoV-2.

10. The method of claim 1, wherein the at least one type of anti-SARS-CoV-2 antibody detected is an anti-SARS-CoV-2 IgA antibody, an anti-SARS-CoV-2 IgM antibody, an anti-SARS-CoV-2 IgG antibody or any combination thereof.

11. The method of claim 1, wherein none of the at least two different types of microparticle reagents, at least one first detection reagent, and the at least one second detection reagent include any anti-species antibodies.

12. The method of claim 1, wherein the at least two different types of microparticle reagents, the at least one of the first detection reagent, the at least one second detection reagent or both the at least one first detection reagent and at least one second detection reagent is an anti-species IgA (e.g., anti-human-IgA IgG) antibody, an anti-species IgG (e.g., anti-human-IgG IgG) antibody, an anti-species IgM (e.g., anti-human-IgM IgG) antibody, or any combination thereof.

13. The method of claim 1, wherein the method further comprises (a) monitoring the subject for SARS-CoV-2 IgA, SARS-CoV-2 IgG, SARS-CoV-2 IgM antibodies and/or SARS-CoV-2 antigen; (b) treating the subject for SARS-CoV-2 infection; (c) monitoring the subject for SARS-CoV-2 IgA, SARS-CoV-2 IgG, SARS-CoV-2 IgM antibodies and/or SARS-CoV-2 antigen and treating the subject for SARS-CoV-2; or (d) treating the subject for SARS-CoV-2 infection and monitoring the subject for SARS-CoV-2 IgA, SARS-CoV-2 IgG, SARS-CoV-2 IgM antibodies and/or at least one type of SARS-CoV-2 antigen.

14. The method of claim 13, wherein SARS-CoV-2 infection is determined by detecting the presence of SARS-CoV-2 viral RNA using polymerase chain reaction, detecting presence of a SARS-CoV-2 viral antigen, or a combination thereof.

15. The method of claim 1, wherein the method is performed in from about 5 to about 20 minutes, and optionally is performed in about 15 to 30 minutes.

16. The method of claim 1, wherein the method further comprises use with at least one calibrator reagent, at least one control reagent, or at least one calibrator reagent and at least one control reagent.

17. The method of claim 1, wherein the method is selected from the group consisting of an immunoassay or a clinical chemistry assay.

18. The method of claim 1, wherein the method is performed using single molecule detection, a lateral flow assay, or a point-of-care assay.

19. The method of claim 1, wherein the method is adapted for use in an automated system or a semi-automated system.
